(19) United States
(12) Patent Application Publication
(10) Pub. No.: US 2022/0411757 A1
(43) Pub. Date: Dec. 29, 2022

(54) Methods for Generating Cardiac Fibroblasts

(71) Applicant: Wisconsin Alumni Research Foundation, Madison, WI (US)

(72) Inventors: Sean Palecek, Verona, WI (US); Martha Floy, Madison, WI (US)

(21) Appl. No.: 17/836,693

(22) Filed: Jun. 9, 2022

Related U.S. Application Data
(60) Provisional application No. 63/209,429, filed on Jun. 11, 2021.

(51) Int. Cl. C12N 5/077; C07K 14/50; G01N 33/50; G01N 33/487

(52) U.S. Cl. CPC C12N 5/0656; C07K 14/503; G01N 33/5044; G01N 33/48728; C12N 5/0657

(57) ABSTRACT

The disclosure generally relates to methods for generating cardiac fibroblast cells from epicardial cardiac progenitor cells, populations of cardiac fibroblast cells and uses thereof.

This application claims the benefit of U.S. Provisional Patent Application No. 63/209,429, filed Jun. 11, 2021, the disclosure of which is explicitly incorporated herein in its entirety by reference.

### STATEMENT REGARDING FEDERALLY SPONDERED RESEARCH

This invention was made with government support under EB007534 awarded by the National Institutes of Health and under 1648035 and 1743346 awarded by the National Science Foundation. The government has certain rights in the invention.

### FIELD OF THE DISCLOSURE

The disclosure generally relates to methods for generating cardiac fibroblast cells from epicardial progenitor cells, populations of cardiac fibroblast cells and uses thereof.

### BACKGROUND

Cardiovascular disease accounts for one in three deaths in the United States and 48% of all adults over 20 years old suffer from cardiovascular disease. Fibroblasts are one of the most prevalent cardiac cell types and estimates suggest they comprise approximately 20-60% percent of the total heart cells, in contrast to cardiomyocytes (CMs) which comprise about 30% of the heart. Fibroblasts in many organs serve as support cells by producing extracellular matrix (ECM) and secreting paracrine factors. Under stress associated with injury and disease, fibroblasts produce excess ECM, inflammatory cytokines, and contribute to scar tissue.

During heart development, cardiac fibroblasts (CFBs) arise from four progenitor populations: epicardial cells, endocardial cells, neural crest cells, and second heart field progenitors. Epicardial cells line the surface of the heart at mouse embryonic day E9.5 and undergo epithelial-to-mesenchymal transition to generate epicardial-derived cells that migrate into the myocardium around E12.5. Lineage tracing studies of Tbx18 and Tcf21-expressing cells have shown that epicardial-derived fibroblasts are primarily located in the ventricles but also contribute to the atrioventricular valves in many model organisms including zebrafish, quail, and mice where they comprise approximately 80% of all CFBs. Conversely, endocardial cells which line the inside of the heart chambers arise at E8.0 and primarily contribute to fibroblast populations in the ventricles and septum in mice starting around E9.5. Neural crest cells contribute primarily to fibroblasts in the coronary trunk and aorta as demonstrated by Pax3 lineage tracing in mice around E9.5. Second heart field progenitors which are present by E8.0 are also thought to differentiate into fibroblasts in the outflow tract as well as the dorsal mesenchymal protrusion, important for atrial septation, thus contributing to atrial fibroblasts. However, despite the results of lineage tracing studies, it is not yet clear whether or how the developmental origin and corresponding developmental timeframe of CFBs influences their subsequent phenotype and function.

In attempts to better characterize cardiac cell diversity, researchers have used single cell transcriptomic profiling to classify and compare populations of cells at different developmental stages. Non-biased clustering of cells of mouse and human hearts have identified 4-6 different fibroblast populations which can be discriminated from other cardiac cell types by high expression of ECM genes and little to no expression of genes encoding sarcomeric proteins. In addition, spatiotranscriptomic approaches, such as fluorescence in situ hybridization (FISH) to target clusters identified from single cell transcriptomics, have been used to trace fibroblast populations to different regions of the developing human heart. A recent single cell RNA sequencing study of adult human hearts found that CFBs in the atria differentially express genes including CNTN4 and NAV2 while CFBs from the ventricles express genes including BMPER and ADCY1. Additionally, fibroblasts in the left and right sides of the heart differentially expressed genes with links to fibrosis, including CLIP and ITGBL1. Another transcriptomic study identified differences in collagen isoforms and ECM-related transcripts between heart chambers.

Human pluripotent stem cells (hPSCs) offer a tool to differentiate cells through developmentally relevant stages and systematically study human CFB function. Over the past ten years, methods have been developed to differentiate hPSCs to cardiac cell types through T³⁰ primitive streak-like mesoderm to MESP1⁺ and GATA4⁺ cardiac mesoderm and further into cardiac progenitors by modulating Wnt signaling. To differentiate hPSCs into second heart field fibroblasts, cardiac mesoderm progenitors were treated with FGF2 to generate NKX2-5⁺, HAND2⁺ and transient TBX1⁺ and CXCR4⁺ second heart field progenitors to TE7⁺POSTN⁺MF20⁻ CFBs. Alternatively, hPSCs can be differentiated to epicardial cells by modulation of Wnt signaling using either recombinant protein WNT3A or small molecule CHIR99021 and further treated with FGF2 to achieve POSTN⁺ CFBs. These EpiC-FBs have been shown to respond to pro- and anti-fibrotic drugs and have been used to study paracrine signaling implicated in fibrogenesis. Furthermore, tissue constructs containing hPSC-CMs and hPSC-epicardial cells implanted in a mouse myocardial infarction, resulted in epithelial-to-mesenchymal-transition (EMT) of epicardial cells to CFBs, improved engraftment, and improved ejection fraction one month later compared to CM monoculture grafts.

Human CFBs hold promise for a variety of applications. Although cardiac cell therapy is actively being investigated by many groups around the world, clinically tested cell preparations have proven disappointing. CFBs can be obtained from animal hearts for research, but human-specific biology is most accurately reflected by human CFBs. Primary, viable human CFBs are difficult to obtain from human cardiac samples obtained by heart biopsies, cardiac surgery, or at autopsy. In addition, primary CFBs can be passaged only a limited number of times before senescence. Thus a robust and reliable source of human CFs is needed for cardiovascular research and therapeutic applications. Accordingly, there is a need in the art for efficient and cost-effective protocols for generating functional cardiac fibroblasts.

### SUMMARY

Provided herein is a method for generating a population of CFBs, the method comprising: culturing epicardial progenitor cells in a culture medium comprising a fibroblast growth factor, whereby a cell population comprising CFBs is obtained.

Also provided herein is a population of CFBs produced by a method comprising: culturing epicardial progenitor cells in a culture medium comprising a fibroblast growth factor, whereby a cell population comprising CFBs is obtained.

Also provided herein is a method of screening a test agent, the method comprising: co-culturing a population of CFBs and the test agent; measuring a functional parameter of the contact co-culture; and comparing the functional parameter to that parameter measured in a co-culture which has not been contacted with the test agent, wherein modulation of the functional parameter after contact with the test agent indicates the test agent is a candidate therapeutic agent.

Also provided herein is a kit for differentiating epicardial progenitor cells into CFBs, the kit comprising: (i) a culture medium suitable for differentiating epicardial progenitor cells into CFBs; (ii) a fibroblast growth factor; and (iii) instructions describing a method for generating CFBs, the method employing the culture medium and the fibroblast growth factor.

### BRIEF DESCRIPTION OF THE DRAWINGS

FIGS. 1A-1B show comparison of fibroblast markers and cardiac transcription factors. FIG. 1A is a schematic diagram of hPSC differentiation to epicardial-derived and second heart field progenitor-derived cardiac fibroblasts. FIG. 1B shows qPCR analysis of cardiac transcription factor expression in EpiC-FB at P1 and SHF-FBs at P3 relative to GAPDH. The y-axis corresponds to relative fold change, 2\hat{ }(−ΔΔCt). Each color represents a different differentiation and each dot represents the average of two technical replicates. Samples having expression below the limit of detection are reported as not detected (N.D.). Statistics are *P<0.05 and **P<0.01 using Student's t-test comparing averages of three well replicates from three or four independent SHF-FB and EpiC-FB differentiations.

FIGS. 2A-2C show molecular characterization of human CFB and maintenance of EpiC-FB. FIG. 2A are example flow gating plots and immunocytochemistry. Undifferentiated hPSCs are in red, no primary control is in blue, EpiCs are in orange, and EpiC-FBs are in green. Scale bar is 100 μm. FIG. 2B is flow cytometry analysis of fibroblast markers (FSP1, TE7, CD90, and VIM), an epicardial marker (WT1) and smooth muscle cell marker (Calponin) expression. Samples include undifferentiated hPSCs, EpiCs, dFBs, SHF-FBs, and EpiC-FBs. Averages of three differentiations each with three technical replicates ±SEM are shown, *P<0.05 and **P<0.01 using ANOVA followed by Tukey's post hoc test. FIG. 2C shows the growth rate of EpiC-FB over 60 days and maintenance of TE7 expression by flow cytometry in H9 hPSC line and 19-9-11 hiPSC line.

FIGS. 3A-3B show RNA sequencing transcriptomic analysis of hPSC-CFBs. FIG. 3A is a volcano plot comparing EpiC-FB at P1 and SHF-FB at P3. FIG. 3B is a GSEA KEGG Pathway Enrichment plot on a preranked list of genes based on −log₁₀(P)*sign*log₂(FC).

FIGS. 4A-4B show heat maps showing further RNA sequencing transcriptomic analysis. FIG. 4A is a heat map showing hierarchical clustering of cardiac transcription factors. FIG. 4B is a heat map showing hierarchical clustering of ECM related genes.

FIGS. 5A-5C shows K-means clustering of highly differentially expressed genes identified by RNA sequencing. FIG. 5A is a heat map with K-means clustering of top 1,000 differentially expressed genes. FIG. 5B shows gene ontology of genes enriched in primary CFB or hPSC-CFB samples. ES is enrichment score [−log₁₀(P)]. FIG. 5C is qPCR analysis of cardiac transcription factor expression in EpiC-FB at P1 and SHF-FBs at P1 relative to GAPDH. The y-axis corresponds to relative fold change, 2\hat{ }(−ΔΔCt). Each color represents an independent differentiation and each dot represents the average of two technical replicates. Samples having expression below the limit of detection are reported as not detected (N.D.). Statistics are *P<0.05 and **P<0.01 using Student's t-test comparing the averages of three well replicates from three independent SHF-FB and EpiC-FB differentiations.

FIGS. 6A-6B show mass spectrometry proteomics comparing decellularized matrix deposited by EpiC-FBs, SHF-FBs, dermal fibroblasts (dFBs), primary adult CFBs (aCFBs), and primary fetal CFBs (fCFBs). FIG. 6A illustrates pie charts displaying fractional compositions of extracellular matrix components. FIG. 6B is a heat map comparing matrix and secreted factors from fibroblast populations.

FIGS. 7A-7B show matrix protein deposition by CFB populations. FIG. 7A shows immunocytochemistry of EpiC-FBs and decellularized ECM for Hoescht and fibronectin. Scale bar is 200 FIG. 7B shows matrix proteins identified by mass spectrometry proteomics comparing decellularized matrix deposited by EpiC-FBs, SHF-FBs, dFBs, aCFBs, and fCFBs. Box and whisker plots comparing expression between fibroblasts. Statistics are ANOVA with Tukey's post hoc test where * is P<0.05 and ** is p<0.01.

FIGS. 8A-8C show secreted factors associated with decellularized FB matrix and secretion of WNT5A. FIG. 8A shows matrix-associated proteins identified by mass spectrometry proteomics comparing decellularized matrix deposited by EpiC-FBs, SHF-FBs, dFBs, aCFBs, and fCFBs. Box and whisker plots comparing expression between fibroblasts. Statistics are ANOVA with Tukey's post hoc test where * is p<0.05 and ** is p<0.01. FIG. 8B shows qPCR comparison of WNT5A expression in EpiC-FB and SHF-FB relative to GAPDH. Averages of three differentiations, each with three replicates ±SEM are shown, *P<0.05 and **P<0.01 using Student's t-test. FIG. 8C is a western blot of WNT5A in EpiC-FB and SHF-FB lysates. Averages of three differentiations, each with three replicates ±SEM normalized to β-actin are shown, *P<0.05 and **P<0.01 using Student's t-test.

FIGS. 9A-9F show fibroblast stress activation akin to myofibroblast SMA activation by addition of TGFβ-1, Angiotensin-II, or serum. Flow cytometry analysis of EpiC-FBs and SHF-FBs treated with FibroGRO or DMEM+10%FBS media (F=FibroGRO, D=DMEM+10%FBS) and small molecule additions of 10 ng/mL TGFβ1, 100 ng/mL TGFβ1, or 1000 ng/mL Angiotensin-II to induce fibroblast activation. Each color represents a different differentiation and each dot represents a well replicate. FIG. 9A shows percentage of cells expressing SMA. FIG. 9B shows median FSC comparison as a relative analysis of cell size. FIG. 9C shows SMA normalized mean fluorescence intensity (MFI) in the SMA+population as a sign of fibroblast activation. Statistics are *P<0.05 and **P<0.01 using two-way ANOVA comparing cell treatments. FIG. 9D shows box and whisker plots of fold change of the percentage of cells expressing SMA, FSC-A, and SMA MFI compared to FibroGRO media condition for each experiment and cell type. Statistics are *P<0.05 and **P<0.01 using 3-way ANOVA controlling for cell treatment and experiment. FIGS. 9E and 9F are example flow gating plots for SMA expression. Controls include EpiCs treated with TGF=1 (smooth muscle cell-like), undifferentiated hPSCs, and fibroblasts with no primary antibody.

FIGS. 10A-10B show SMA staining of activated aCFBs and dFB controls. FIG. 10A shows results from immunocytochemistry analysis of SMA and VIM after treatment with various medias to induce fibroblast activation. Hoechst nuclear counterstain (blue) is also included. Scale bar is 200 μm. FIG. 10B is flow cytometry analysis of dFBs and aCFBs treated with FibroGRO or DMEM+10%FBS media (F=FibroGRO, D=DMEM+10%FBS) and small molecule additions of 10 ng/mL TGFβ1, 100 ng/mL TGFβ1, or 1000 ng/mL Angiotensin-II to induce fibroblast activation. Median FSC comparison as a relative analysis of cell size. SMA expression as a sign of fibroblast activation. Each color represents an independent differentiation and each dot represents a well replicate. Statistics are *P<0.05 and **P<0.01 using ANOVA comparing media formulations.

FIGS. 11A-11F show mineralization of CFBs. Fibroblasts were treated with osteogenic media for 4 weeks to induce calcification and mineralization. FIG. 11A shows relative ALP activity in dFBs, fCFBs, aCFBs, EpiC-FBs, and SHF-FBs. Red bars represent control media (αMEM+10%FBS) and blue bars represent osteogenic media (αMEM+10%FBS+50 mg/L L-ascorbic acid 2-phosphate sesquimagnesium salt hydrate+10 mM β-glycerophosphate disodium salt hydrate+10 nM dexamethasone). Plotted are the mean±SEM of three well replicates, *P<0.05 and **P<0.01 comparing control media and osteogenic media for each cell type using Student's t-test. The graph is representative of four independent differentiations. FIG. 11B shows percentage change in ALP activity of fibroblasts in osteogenic media compared to control media. Plotted are mean percentage change of three well replicates with error bars calculated by propagation of error, *P<0.05 and **P<0.01 comparing between cell types using ANOVA. The graph is representative of four independent differentiations. FIG. 11C shows relative ALP activity of CFBs treated with αMEM+10%FBS for four weeks, *P<0.05 and **P<0.01 comparing between cell types using ANOVA. FIG. 11D shows Alizarin red staining to depict fibroblast mineralization in osteogenic media. Scale bar is 100 μm. FIG. 11E shows quantification of Alizarin red staining. Plotted are mean percentage change of three well replicates with error bars calculated by propagation of error, *P<0.05 and **P<0.01 comparing between cell types using ANOVA. The graph is representative of three independent differentiations. FIG. 11F shows percentage change in Alizarin red staining of fibroblasts in osteogenic media compared to control media. Plotted are mean percentage change of three well replicates with error bars calculated by propagation of error, *P<0.05 comparing between cell types using ANOVA. The graph is representative of three independent differentiations. FIG. 11G shows Alizarin red staining of CFBs treated with αMEM+10%FBS for four weeks, *P<0.05 and **P<0.01 comparing between cell types using ANOVA.

FIGS. 12A-12C show hPSC-cardiac microtissue formation and calcium handling. Enriched hPSC-CMs were seeded alone or in combination with EpiC-FBs, SHF-FBs, or with fCFBs (3:1 ratio) at 2000 cells per microwell. FIG. 12A shows bright field images of microtissues (i). 1 day later, these heterotypic cell mixtures had robustly self-assembled into 3D spheroids while the CM-only cells had not (ii-in microwells; iii-immediately after removing from microwells). Heterotypic cardiac microtissues compacted over the next few days (iv) and remained stable throughout 10 days of culture (v). Scale bar is 50 μm. FIG. 12B shows immunocytochemistry of sectioned aggregates for DAPI, cTnT, and VIM. Scale bar is 100 μm. FIG. 12C shows GCaMP fluorescence of calcium flux in cardiac microtissues (top) and definitions of calcium handling parameters (bottom). Cardiac microtissues were subjected to 1 Hz electrical field stimulation and calcium transient amplitude, time-to-peak, and stroke velocities were quantified for each microtissue condition. *p<0.05, **p<0.01, ****p<0.0001.

### DETAILED DESCRIPTION

The disclosure generally relates to methods for generating cardiac fibroblast cells from epicardial progenitor cells, populations of cardiac fibroblast cells, and uses thereof.

All publications, including but not limited to patents and patent applications, cited in this specification are herein incorporated by reference as though set forth in their entirety in the present application.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

In some embodiments provided herein are methods for generating populations of cardiac fibroblast cells, the methods comprising: culturing epicardial progenitor cells in a culture medium comprising a fibroblast growth factor, whereby a cell population comprising cardiac fibroblast cells is obtained.

In particular embodiments, the epicardial progenitor cells are human.

The term “cardiac fibroblast” refers to cells of the cardiac fibroblast lineage. Cardiac fibroblasts are characterized and identified by expression of biomarkers including Islet-1 (ISL1), and fibroblast markers VIM (vimentin) and CD90 as well as staining positive with the TE7 anti-fibroblast antibody

In particular embodiments, the epicardial progenitor cells are cultured in a serum-free culture medium including a fibroblast growth factor (FGF) for differentiation. In particular embodiments, the epicardial progenitor cells are cultured in xeno-free medium including a fibroblast growth factor (FGF) for differentiation. In particular embodiments, the epicardial progenitor cells are cultured in chemically defined medium including a fibroblast growth factor (FGF) for differentiation. The terms “chemically-defined culture conditions” and “fully-defined conditions” indicate that the identity and quantity of each medium ingredient is known and the identity and quantity of any supportive surface is known. In some embodiments, cardiac fibroblast cells are obtained after about 9-12 days in culture (i.e., about 9, 10, 11 or 12 days in culture). In one embodiment, epicardial progenitor cells are cultured for 10 days. In one embodiment, epicardial progenitor cells are cultured for 15 days.

The term “fibroblast growth factor” or “FGF” refers to any of the members of a family of growth factors involved in angiogenesis, wound healing, and embryonic development. There are several different FGF subfamilies, the member ligands of which include FGF1-FGF23. Of the known FGF ligands, all show some degree of overlap of receptor binding, with the exception of FGF11-FGF14. In some embodiments, FGF used in methods as set forth herein is bFGF/FGF2, FGF4, FGF8, or FGF10 and mixtures thereof.

In some embodiments, bFGF concentrations in medium ranges from about 1 ng/mL to about 1000 ng/mL. For example, bFGF concentrations may range from about 10 ng/mL to about 100 ng/mL, or from about 20 ng/mL to about 200 ng/mL, or about 30 ng/mL to about 300 ng/mL, or about 40 ng/mL to about 400 ng/mL, or about 50 ng/mL to about 500 ng/mL, or about 60 ng/mL to about 600 ng/mL, or about 70 ng/mL to about 700 ng/mL, or about 80 ng/mL to about 800 ng/mL or about 90 ng/mL to about 900 ng/mL. In some embodiments, bFGF concentrations in a medium is about 5 ng/mL.

The methods provided herein produce populations of pluripotent stem cell-derived CFBs, where the population is a substantially pure population of CFBs. The term “substantially pure” refers to a population of cells that is at least about 75% pure, with respect to CFBs making up a total cell population. In other words, the term “substantially pure” refers to a population of CFBs of the present disclosure that contains fewer than about 20%, fewer than about 10%, or fewer than about 5% of non-cardiac fibroblast cells (e.g., cardiomyocytes, smooth muscle cells, endothelial cells) when directing differentiation to obtain cells of the CF lineage. The term “substantially pure” also refers to a population of CFs of the present invention that contains fewer than about 25%, about 10%, or about 5% of non-CFs in an isolated population prior to any enrichment, expansion step, or further differentiation step. Typically, a population including CFBs obtained by the disclosed methods comprises a very high proportion of CFBs. In some embodiments, the cell population comprises about 50% to about 99% CFBs, e.g., about 52%, 55%, 67%, 70%, 72%, 75%, 80%, 85%, 90%, 95%, 98%, or another percent of CFBs from about 50% to about 99% CFBs.

CFBs can be identified by the presence of one or more CFB markers. Useful gene expression or protein markers for identifying CFBs include, without limitation, GATA4, HAND2, HEY1, ISL1, NKX2.5, and WT1 (Wilms tumor protein), VIM, CD90, FSP1, POSTN, and PDGFRB. Preferably, practice of methods disclosed herein yields a cell population, at least 90% (e.g., at least 90%, 93%, 95%, 96%, 97%, 98%, 99% or more) of which are CFBs positive for a fibroblast marker (anti-human fibroblast antibody, clone TE-7, Millipore) and cardiac transcription factor GATA4, and negative for cardiomyocyte markers including myosin heavy chain isoforms (MYH6 and MYH7) and cTnT, and negative for smooth muscle markers including calponin. Molecular markers of CFBs can be detected at the mRNA expression level or protein level by standard methods in the art.

In some embodiments, methods disclosed herein yield a cell population, at least 85%, at least 90%, at least 95% or at least 99% of which are cardiac fibroblast cells positive for expression of one or more the markers TBX2, TBX18 and TBX20. Molecular markers can be detected as expressed mRNA or protein by conventional methods in the art.

In some embodiments provided herein are populations of cardiac fibroblast cells produced by the methods disclosed herein. In particular embodiments, these population of cells are positive for expression of one or more of the markers TBX2, TBX18 and TBX20.

In some embodiments, provided herein are compositions and methods for expanding a self-renewing population of CFBs for at least 60 days. In some embodiments, provided herein are compositions and methods for expanding a self-renewing population of CFBs capable of being passaged at least 10, 11, 12, 13, 14 or 15 times, wherein these cells are non-senescent and are not immortalized. For example, CFBs maintain expression levels of TE-7, vimentin, and/or for GATA4 for about 60 days. Therefore, provided herein is an expandable source of functional human CFB cells.

In particular embodiments disclosed herein are methods of screening a test agent, the methods comprising: co-culturing a population of cardiac fibroblast cells prepared according to methods disclosed herein with the test agent; measuring a functional parameter of the contacted co-culture; and comparing the functional parameter to that parameter measured in a co-culture which has not been contacted with the test agent, wherein modulation of the functional parameter after contact with the test agent indicates the test agent is a candidate therapeutic agent. In some embodiments, a test agent may be characterized as having cardiac toxicity when the test agent modulates the functional parameter away from physiologically acceptable conditions. In some embodiments, test agents can be screened for influence on prolongation of the QT interval, wherein test agents that prolong the QT interval will be considered agents with a potential to induce drug-induced long QT syndrome.

“Test agent” refers to a molecule assessed for its ability to alter a specific phenotypic endpoint. Examples of test agents include, but are not limited to, (i) organic compounds of molecular weight less than about 600 daltons; (ii) nucleic acids; (iii) peptides (including stapled peptides); (iv) polypeptides; and (v) antibodies or antigen-binding fragments thereof. In some embodiments, the test agent is an antifibrotic therapeutic agent.

Functional parameters can include electrical impulse propagation pattern, conduction velocity, or action potential duration. An electrical impulse propagation pattern can be measured using a fluorescent membrane potential dye. Acceleration of conduction velocity after contact with a test agent can indicate the test agent is a candidate therapeutic agent. Prolongation of the action potential duration after contact with the test agent can indicate the test agent is a candidate therapeutic agent. An electrical impulse propagation pattern can be measured as the fibrillatory or reentry pattern and an increase in the pattern after contact with the test agent indicates the test agent is a candidate therapeutic agent.

In some embodiments, provided herein is a kit for differentiating epicardial progenitor cells into cardiac fibroblasts, the kit comprising: (i) a culture medium suitable for differentiating epicardial progenitor cells into cardiac fibroblasts; (ii) a fibroblast growth factor; and (iii) instructions describing a method for generating cardiac fibroblasts, the method employing the culture medium and the fibroblast growth factor.

Without limiting the disclosure, a number of embodiments of the disclosure are described below for purpose of illustration.

### EXAMPLES

The Examples that follow are illustrative of specific embodiments of the disclosure, and various uses thereof. They are set forth for explanatory purposes only and should not be construed as limiting the scope of the disclosure in any way.

Materials and Methods

Maintenance of hPSCs

Human pluripotent stem cells (hPSCs) were maintained on Matrigel (Corning)-coated plates in mTeSR1 (STEMCELL Technologies) according to previously published methods (Lian, et. al., 2013, Nat. Protoc. 8, 162-175). At 80-90% confluency hPSCs were passaged with Versene to maintain colonies. For this study, hESC line H9 (WiCell) and H9-7TGP (Palecek Lab) and hiPSC lines WTC-CAAX-RFP (Allen Institute), WTC11-GCaMP (Gladstone), and 19-9-11 (WiCell) were used.

Cardiac Progenitor Cell Differentiation via Modulation of Canonical Wnt Signaling

As previously published in the GiWi protocol to derive cardiac progenitors, hPSCs were singularized with Accutase at 37° C. for 5 minutes, quenched in DMEM/F12, and centrifuged at 200 g for 5 minutes (Lian, et. al., 2013, Nat. Protoc. 8, 162-175). hPSCs were seeded at 100,000-600,000 cells/cm² in mTeSR1 supplemented with 5 μM ROCK inhibitor Y-27632 (Selleckchem) (day -2) for 24 hours. The following day (day -1), cells were treated with fresh mTeSR1. At day 0, cells were treated with 6-15 μM CHIR99021 (Selleckchem) in RPMI1640 supplemented with B27 minus insulin (RPMI/B27⁻) media. Exactly 23-24 hours later, media was changed to fresh RPMI/B27⁻ (day 1). At day 3, 504 IWP2 (Tocris) was added to 1:1 conditioned media to fresh RPMI/B27⁻ media. At day 5, cells were treated with RPMI/B27⁻ media. At day 6, cardiac progenitors were either frozen in cryomedia (60% DMEM/F12, 30% FBS, 10% DMSO) or singularized for further differentiation.

Epicardial Cell Differentiation via Activation of Canonical Wnt Signaling

Following a previously published protocol for epicardial differentiation, day 6 cardiac progenitors were either singularized in Accutase at 37° C. for 10 minutes or thawed from cyro and seeded onto a gelatin (Sigma-Aldrich) or Matrigel-coated cell culture plate at 20,000-80,000 cells/cm² (approximately a 1:3 or 1:12 split) in LaSR basal media (500 mL advanced DMEM/F12 (Life Technologies) with 0.06 g/L L-ascorbic acid 2-phosphate sesquimagnesium salt hydrate (Sigma-Aldrich) and 6.25 mL GlutaMAX) supplemented with 5 μM ROCK inhibitor Y-27632 (Bao, et al., 2017, Nat. Protoc. 12, 1890-1900). At days 7 and 8, cells were treated with fresh LaSR basal media supplemented with 3 μM CHIR99021. At days 9, 10, and 11, cells were treated with fresh LaSR basal media. At day 12, epicardial cells were singularized with Accutase for 5 minutes at 37° C. and either cryopreserved for later use or replated in LaSR basal media supplemented with A8301 (R&D Systems) and 54 μM ROCK inhibitor Y-27632. Subsequently, epicardial cells were treated with LaSR supplemented with A8301 daily until they became 90-100% confluent. Epicardial cells were then passaged using Versene into fresh LaSR basal media supplemented with 0.5 μM A8301 without ROCK inhibitor Y-27632 to maintain colonies, prevent further differentiation, and improve attachment for up to five passages. Alternatively, cells were frozen in cryomedia (60% DMEM/F21, 30% FBS, 10% DMSO). Differentiations were validated to have at least 90% WT1 positive cells by flow cytometry.

Epicardial Fibroblast Differentiation via bFGF Signaling

When epicardial cells reached approximately 100% confluency, they were treated with LaSR supplemented with 5 ng/mL bFGF (Waisman Biomanufacturing) daily for 10 days. At this point, EpiC-FBs were cryopreserved or passaged at 7,000 cells/cm² or approximately 1:3-1:6 split with Accutase into FibroGRO (EMD Millipore) media on a cell culture treated plate (FibroGRO basal media with manufacturers supplements, GlutaMAX supplemented for Glutamine, and 2% FBS). Media was changed every two days until fibroblasts reached approximately 80-90% confluency when they were passaged with Accutase. Differentiations were validated to have be at least 80% double positive for TE-7 and VIM by flow cytometry. All experiments were performed between P1 and 5 unless otherwise noted.

Epicardial Smooth Muscle Cell Differentiation via TGFβ Signaling

When epicardial cells reached approximately 100% confluency, they were treated with LaSR supplemented with 5 ng/mL TGFβ (Waisman Biomanufacturing) daily for 6 days (Bao, et al., Nat. Protoc. 12, 1890-1900 (2017) At this point, smooth muscle cells (SMCs) were used for experiments.

Cardiomyocyte Differentiation

WTC11-GCaMP6f hiPSCs (Mandegar, et al., 2016, Cell Stem Cell. 18, 541-553) were differentiated into CMs following the GiWi protocol (Lian et. al., 2013, Nat. Protoc. 8, 162-175). Briefly, hPSCs were seeded onto Matrigel-coated (80 μg/mL) plates at a density of 3×10⁴ cells/cm² in mTeSR medium with 10 μM Rock inhibitor. Once cells reached 100% confluence (˜3 days), medium was changed to RPMI/B27⁻supplemented with 12 μM CHIR99021 (Day 0). Exactly 24 hours later, cells were fed with fresh RPMI/B27⁻ and on day 3, medium was changed to RPMI/B27⁻ supplemented with 5 μM IWP2. On days 7, 10, and 13, cells were fed with RPMI1640 with B27 supplement containing insulin (RPMI/B27⁺). On day 15, cells were replated onto Matrigel-coated plates at a density of 2×10⁵ cells/cm² in RPMI/B27⁺ containing 15% FBS and 10 μM Rock inhibitor. Medium was changed to fresh RPMI/B27⁺ on day 16. Enrichment of CMs occurred by feeding cells with lactate purification medium (Tohyama et al., 2013, Cell Stem Cell. 12, 127-137) (no-glucose Dulbecco's Modified Eagle Medium with 1× Non Essential Amino Acids, 1× Glutamax, and 4 mM Lactate) on days 17 and 20, and cells were returned to RPMI/B27⁺ on day 23 and used for cardiac microtissues between days 25 and 28.

Second Heart Field Fibroblast Differentiation

Second heart field CFBs were differentiated following the GiFGF protocol as previously published (Zhang et al., 2019, Nat. Commun. 10, 2238). When hPSCs maintained in mTeSR1 reached approximately 90-100% confluency, they were treated with 6-15 μM CHIR99021 in RPMI/B27⁻ media (day 0). Exactly 23-24 hours later, media was changed to fresh RPMI/B27⁻ (day 1). From day 2 to 20, cells were treated with fresh CFB basal media every two days. At day 20, CFBs were singularized with Accutase for 10 minutes at room temperature and cryopreserved or replated on tissue cultured plastic in FibroGRO media at approximately 7,000 cells/cm'. Following this, FibroGRO media was changed every two days until the fibroblasts reached approximately 80-90% confluency when they were passaged with Accutase. Differentiations were validated to have be at least 80% double positive for TE-7 and VIM by flow cytometry. All experiments were performed between P1 and 5 unless otherwise noted.

Primary Fibroblast Cell Culture

Primary human adult dFBs (Lonza), primary human adult ventricular CFBs (Lonza), and primary human fCFBs (Cell Applications) were cultured in FibroGRO media. For these studies, dFB, aCFB, and fCFB were used from a single donor, the key attributes representative of the age group across many genetic backgrounds could be determined. Media was changed every two days until the fibroblasts reached approximately 80-90% confluency when they were passaged with Accutase up to five times.

Immunostaining Analysis

As explained previously, cells were fixed with 4% paraformaldehyde for 10 minutes or ice-cold methanol for 5 minutes at room temperature and then blocked in milk buffer (PBS plus 0.4% Triton X-100 and 5% non-fat dry milk or BSA buffer (PBS plus 0.1% Triton X-100 and 0.5% BSA) for one hour at room temperature. Then, primary antibodies were added, and samples were incubated overnight at 4° C. on a shaker. The following day, cells were washed with PBS and stained with secondary antibodies at room temperature for one hour or overnight at 4° C. on a shaker. Hoescht counterstain was added at 5 μg/mL in PBS for five minutes. For image analysis, an epifluorescence microscope Olympus IX70 or Nikon Ti2 was used. To image thick ECM, fibroblasts were plated on a 35 cm ibidi dish and imaged using a Nikon MR confocal microscope.

Flow Cytometry Analysis

As previously described, cells were singularized with Accutase then fixed with 1% paraformaldehyde for 20 minutes at room temperature and stained with primary antibodies overnight at 4° C. in BSA buffer (PBS plus 0.1% Triton X-100 and 0.5% BSA). The following day, cells were washed and stained with secondary antibodies at room temperature for one hour. At least 10,000 events/sample were collected on a BD Accuri C6 flow cytometer and analyzed using FlowJo. FACS gating was based on a no primary control and negative cell type control.

mRNA Extraction, cDNA Preparation, and qPCR Analysis

Cells were singularized in Accutase, quenched, and centrifuged for 5 minutes at 200 g. Cell pellets were snap-frozen at −80° C. until mRNA extraction. Total RNA was isolated using the RNeasy mini kit (Qiagen) and treated with DNase (Qiagen). Extracted mRNA was stored in nuclease-free water at −20° C. and 1 μg RNA was reverse transcribed into cDNA using the Omniscript Reverse Transcriptase kit (Qiagen) and Oligo(dT)20 Primers (Life Technologies). Real-time quantitative PCR with two technical replicates in 25 uL reactions using PowerUP Syber Master Mix (Applied Biosystems) on an AriaMx Real-Time PCR System at 60° C. (Agilent Technologies). GAPDH and ZNF384 were used as housekeepers and analysis was performed using the AACt method.

Single Cell Sequencing Data Analysis

Count matrices from publicly available single cell sequencing datasets were obtained and selected cells from clusters Asp et al. had previously annotated as fibroblasts or fibroblast-like cells (Asp et al., 2019, Cell 179, 1647-1660.e19). Violin plots were prepared using clusters identified by the authors using the Seurat package (version 3) (Stuart et al., 2019, Cell 177, 1888-1902.e21). Differentially expressed genes amongst the fibroblast clusters were identified and the top ten for each cluster were plotted in a heatmap.

RNA Sequencing

Quality and quantity of RNA samples was first analyzed using Nanochip to confirm presence of 18S and 28S ribosomal RNA with appropriate A260/A280 and A260/A230 ratios. Then, RNA was quantified on an Agilent 2100 Bioanalyzer using Qubit prior to library construction and sequencing. Sequencing libraries were constructed using the Illumina TruSeq Stranded mRNA kit (polyA enrichment). Libraries were sequenced on an Illumina NovaSeq6000. Between 62 and 88 million reads were collected per sample.

Raw FASTQ files were mapped to the human genome (hg38) using RNA STAR (version 2.7.5b) implemented on the Galaxy public server at usegalaxy.org (Afgan et al., 2018, Nucleic Acids Res. 46, W537-W544). Gene-level transcript abundances were calculated using featureCounts (version 1.6.4+galaxy2) in Galaxy.

Differential expression analysis was performed in Galaxy using DESeq2 (version 2.11.40.6+galaxy1) using raw counts as an input. Transcript abundances are presented as transcripts per million (TPM). Hierarchical clustering was performed on TPM using the publicly-available online platform Morpheus. For k-means and GO analysis, we used the top 1000 variable genes and performed an average silhouette approach to identify 6 unique clusters among the genes. K-means cluster analysis was performed using 6 clusters and the genes composing the 6 clusters were imported into Metascape for an express pathway analysis. GO analysis was performed on genes with fold change >2 and p<0.05 GSEA analysis was performed on preranked list of differentially expressed genes based on log2(FC)*log10(p).

Western Blot Analysis

Cells were lysed in RIPA buffer in the presence of Halt Protease Inhibitor Cocktail (ThermoFisher). The BCA assay was used to determine protein concentration. Equal amounts of lysates were loaded on 4 to 12% tris-glycine gels and transferred to nitrocellulose membranes. Membranes were blocked in Tris-buffered saline +0.1% Tween20 (TBST)+5% BSA for 1 hour and incubated with primary antibodies (WNT5A and Beta-actin) overnight at 4° C. on a shaker. The following day, membranes were washed with TBST and incubated with secondary antibodies at 1:5000 in 15 mL antibody solution/blot for 1 hour on a shaker. Blots were washed again. Then, blots were incubated with 12 mL of chemiluminescent substrates (Clarity Western ECL substrate (Bio-Rad) for Beta-actin blots) and (Supersignal West Femto Maximum Sensitivity Substrate (Thermo Fisher) for WNTSA blots) for 5 minutes and analyzed on ChemiDoc XRS+(Bio-Rad). Bands were normalized to Beta-actin loading control.

Osteogenic Induction

Cells were plated at 7,000 k/cm² in a 12 well plate and cultured in aMEM+10%FBS control medium (Life Technologies) or control medium supplemented with 50 mg/L L-ascorbic acid 2-phosphate sesquimagnesium salt hydrate (Sigma), 10 mM β-glycerophosphate disodium salt hydrate (CHEM-IMPEX INT'L INC), and 10 nM dexamethasone (Sigma) osteoinduction. Cells were treated for 3 or 4 weeks with media changes every two days.

ALP Activity Assay

To normalize between samples, the BCA assay was used to quantify protein concentration and equal amounts of protein were used for the Alkaline phosphatase diethanolamine activity kit (Sigma-Aldrich). Two technical replicates were performed per sample and absorbance (410 nm) was measured on a Tecan M100 plate reader.

Alizarin Red Staining and Quantification

After four weeks in osteogenic medium or control medium, cells were fixed in 4% paraformaldehyde for 10 minutes and washed with DI water. Samples were stained with 0.5 mL/well of 40 mM Alizarin red (Sigma) at room temperature on a shaker for thirty minutes. Then, samples were washed four times with 1 mL/well DI water and imaged on an EVOS XL Core Imaging System. To quantify Alizarin red staining, samples were treated with 300 μL/well 10 w/v% cetylpyridinium chloride (Sigma-Aldrich) at room temperature on a shaker for one hour. 150 μL was transferred into 2 wells of a 96 well plate (for technical replicates) and absorbance (560 nm) was measured on a Tecan M100 plate reader.

High Density Fibroblast Culture Decellularization

High density fibroblast culture and decellularization protocols were modified from previous methods (Zhang et al., 2019, Nat. Commun. 10, 2238; Schmuck et al., 2014, Cardiovasc. Eng. Technol. 5, 119-131). Fibroblasts were seeded at 7,000 cells/cm² and cultured in FibroGRO media for 10 days without passaging. At day 10, cells were decellularized using a protocol adapted from Chen et al. (1978, Cell. 14, 377-391) and Harris et al. (2018, Methods Cell Biol. 143, 97-114). Briefly, cells were washed with PBS and then wash buffer 1 (100 mM Na₂HPO₄, 2 mM MgCl₂, 2 mM EDTA). Then, they were lysed in buffer (8 mM Na₂HPO₄, 1% triton) and incubated at 37° C. for three hours with fresh lysis buffer added after each hour. Finally, matrix was washed with wash buffer 2 (100 mM Na₂HPO₄, 300 mM KC1) and washed with DI water. Plates were dried overnight in a sterile environment and then stored at −20° C. until further sample processing.

High Density Fibroblast Culture Mass Spectrometry Sample Preparation

Decellularized high density fibroblast cultures were prepared for trypsinization by removing plates −20° C. for 20 minutes until they reach room temperature (RT). The decellularized protein was dissolved in 75 μL of 6M urea with 3.75uL of 200 mM dithiothreitol (DTT) and incubated for 1-hour at RT. 15 μL of 200 mM iodoacetamide was added into the existing solution in each well and mixed thoroughly followed by a 1-hour incubation at RT in the dark. An additional 15 μL of DTT was added to each well and mixed thoroughly followed by a 1-hour incubation at RT in the dark. The solution was quenched with 340 μL of 1 mM CaCl₂ and the pH was adjusted to 7.8-8.7 with NaOH for optimal trypsin activity. Samples were trypsinized for 24 hours at 37° C. with 5 μL of 1 μL Trypsin Gold, Mass Spectrometry Grade (Promega). The following day, the peptide solution was removed from the well plate and placed in an Eppendorf° LoBind microcentrifuge tube, frozen at −80° C. for at least 3 hours and lyophilized overnight.

Protein purification was done using the ZipTip®_{C18} (Millipore Sigma) protocol as follows. Lyophilized samples were reconstituted in reconstitution solution (5:95 Acetonitrile (ACN):H2O, 0.1% TFA). Sample pH was then adjusted to a pH<3 with 10% TFA. ZipTip®_{C18} were hydrated by aspirating and expelling hydration solution (50:50 ACN:H₂O, 0.1% TFA) from the ZipTip®_{C18} twice, followed by wash solution (0.1% TFA in H₂O) twice. Samples were loaded into the ZipTip®_{C18} by aspirating and expelling the reconstituted sample from the ZipTip®_{C18} 6-times. Samples were desalted by washing 3-times with wash solution. The purified peptides were then eluted into an Eppendorf® LoBind microcentrifuge tube containing elution solution (60:40 ACN:H20, 0.1% TFA). The eluted samples were frozen, lyophilized and stored at −80° C. until further analysis.

Mass Spectrometry Data Acquisition, Processing and Analysis

Purified peptides from decellularized high density fibroblast cultures were reconstituted and analyzed using 1D capillary mass spectrometry on the Thermo Orbitrap Velos. Using Proteome Discover™ Software the raw mass spectrometry data was run through the human UniPprot database for both cellular and extra cellular proteins. Proteins detected from cellular debris were excluded. Proteins with a sum of PEP score below 2 were also excluded to avoid false positives for protein detection. Molar percent of ECM and secreted proteins present were calculated using the exponentially modified protein abundance index (emPAI) as follows: where the emPAIA is the emPAI of the protein of interest and emPAIₜₒₜ is the sum of the emPAI of all ECM and secreted proteins (Ishihama et al., 2005, Mol. Cell. Proteomics MCP. 4, 1265-1272):

$$Protein A Molar Percent = \frac{{emPAI}_{A}}{{emPAI}_{tot}} \times 100$$

Cardiac Microtissue Formation

Lactate-purified CMs, SHF-FBs, EpiC-FBs, and primary human fCFBs (were dissociated with 0.25% Trypsin for 5-10 minutes and then mixed together at a ratio of 3:1 CMs:FBs in RPMI/B27⁺ medium with 10 μM Rock inhibitor. The heterotypic cell mixtures were seeded into 400 μm inverted pyramidal agarose microwells at a density of 2000 cells per microwell and incubated overnight to allow cells to self-assemble into 3D microtissues. 18-24 hours later, the microtissues were removed from the microwells and transferred to low-attachment plates in RPMI/B27⁺ medium. Microtissues were maintained in rotary suspension culture at a density of 8000 tissues per 10 cm plate for 10 days, and fed every 2-3 days with RPMI/B27⁺ medium.

Calcium Imaging

Cardiac microtissues cultured for 10 days were incubated in Tyrode's solution (137 mM NaCl, 2.7 mM KCl, 1 mM MgCl₂, 0.2 mM Na₂HPO₄, 12 mM NaHCO₃, 5.5 mM D-glucose, 1.8 mM CaCl₂) for 30 minutes at 37° C. immediately prior to imaging. A Zeiss Axio Observer Z1 inverted microscope equipped with a Hamamatsu ORCA-Flash 4.0 camera was used for image acquisition. Electrical field stimulation of 1 Hz was applied to the samples by placing electrodes in the Tyrode's bath containing the microtissues (MyoPacer, IonOptix). Calcium transient videos were acquired using Zen Professional software (v.2.0.0.0) at 10 ms exposure and 100 frames per second. Circular regions of interest (65-pixel diameter) were selected at the center of each microtissue and mean fluorescence intensity values were plotted against time. Metrics of calcium transient kinetics, such as amplitude, time-to-peak, upstroke and downstroke velocities, and beat rate, were analyzed using a custom python script.

Sectioning and Staining of Cardiac Microtissues.

Microtissues were fixed in 10% Neutral Buffered Formalin (VWR) for 1 hour at room temperature and embedded in HistoGel Specimen Processing Gel (Thermo Fisher) prior to paraffin processing. Five micron sections were cut and adhered to positively charged glass slides. Slides were deparaffinized with xylene and re-hydrated through a series of decreasing ethanol concentrations (100%, 100%, 95%, 80%, 70%). Epitope retrieval was performed by submersing slides in Citrate Buffer pH 6.0 (Vector Laboratories) in a 95° C. water bath for 35 minutes. Slides were cooled at room temperature for 20 minutes and washed with PBS. Samples were permeabilized in 0.2% Triton X-100 (Sigma-Aldrich) for 5 minutes, blocked in 1.5% normal donkey serum (Jackson Immunoresearch) for 1 hour, and probed with primary and secondary antibodies (1:400) against cTnT and VIM and counterstained with Hoechst (1:10000). Coverslips were mounted with anti-fade mounting medium (ProlongGold, Life Technologies) and samples were imaged on a Zeiss Axio Observer Z1 inverted microscope equipped with a Hamamatsu ORCA-Flash 4.0 camera.

Statistical Analysis

All experiments were conducted using at least three technical replicates (e.g., three 12-wells) from the same differentiation. All experiments were replicated (independent differentiations) at least three times with one replicate in the 19-9-11 hiPSC line and one replicate in the H9 hESC line except where otherwise indicated. Statistical significance was evaluated using Student's t-test, one-way analysis of variance (ANOVA), two-way ANOVA, or three-way ANOVA followed by post hoc tests used for multiple comparisons, Dunnett's test for comparing experimental groups to a control group or Tukey's test for comparing between all experimental groups. P<0.05 was considered statistically significant. Statistical analysis was performed using GraphPad Prism 8.0 or JMP PRO 15 software.

### Example 1:

### Molecular Characterization of hPSC-CFBs Reveals Distinct CFB Signatures

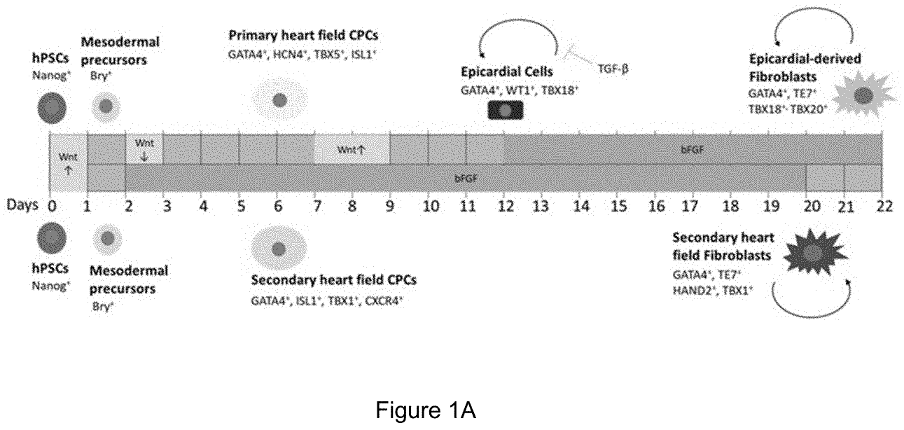

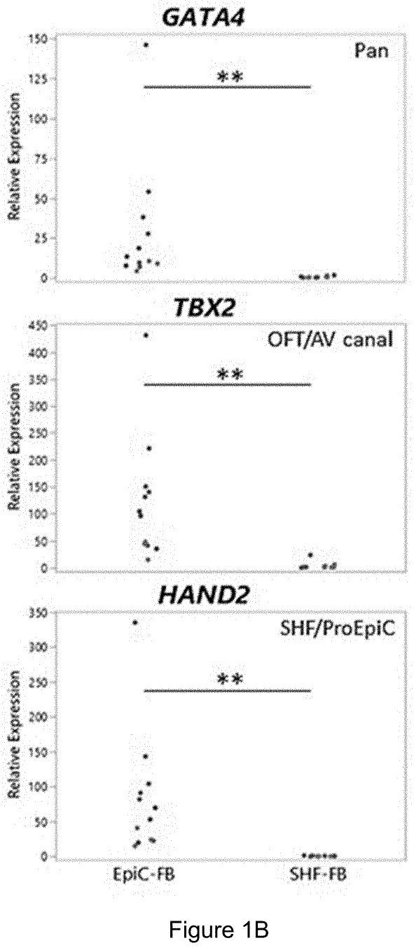

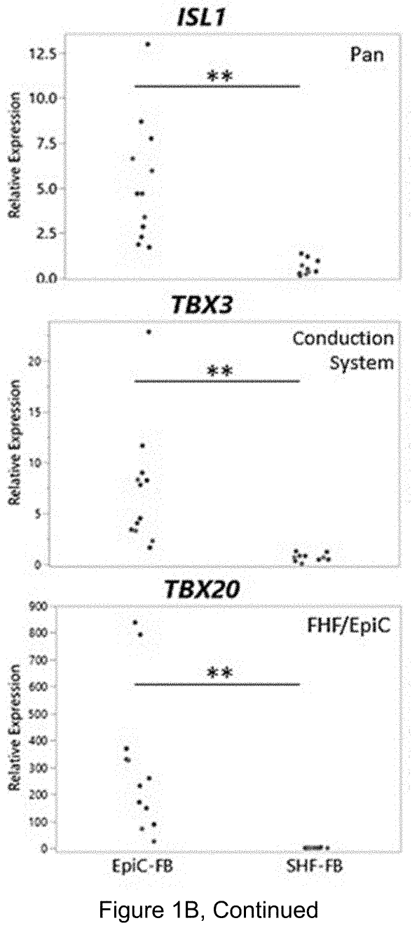

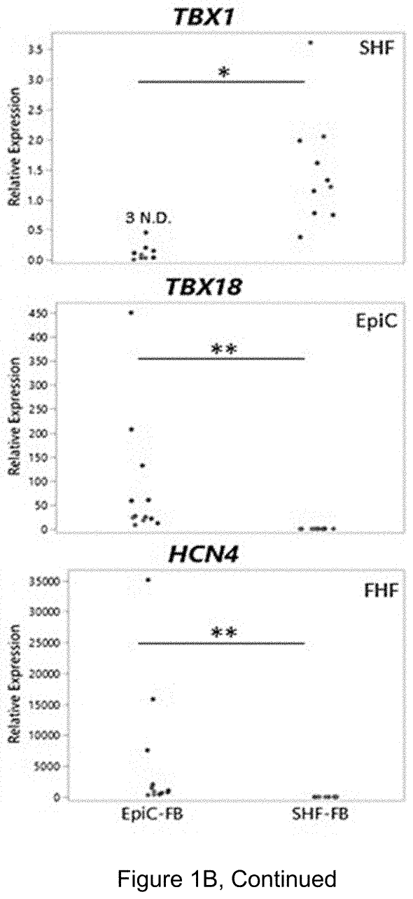

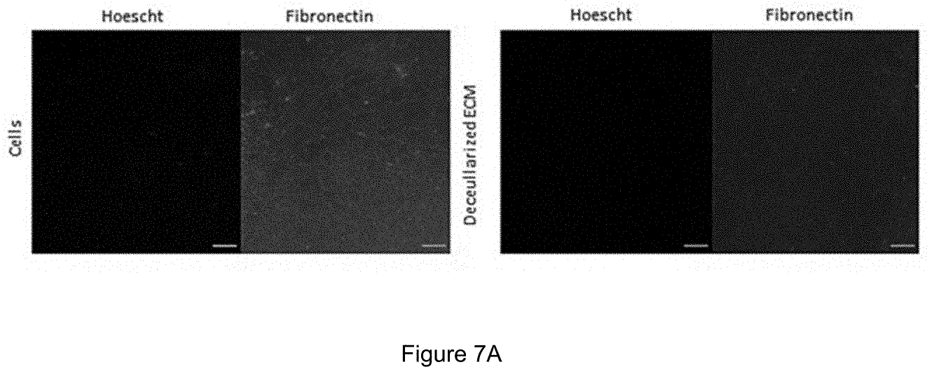

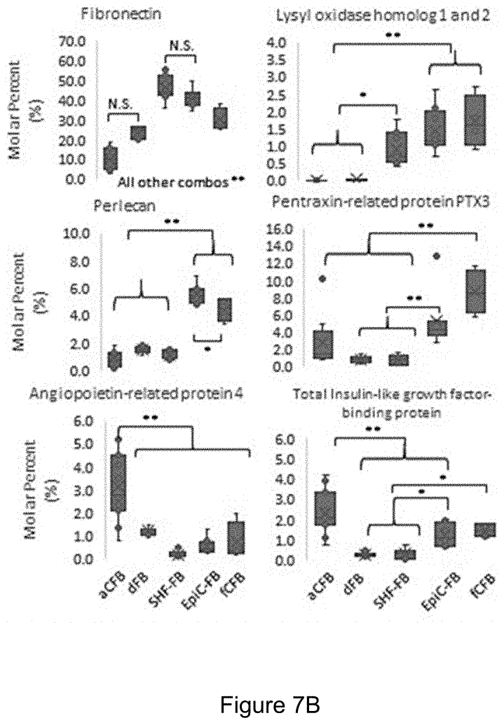

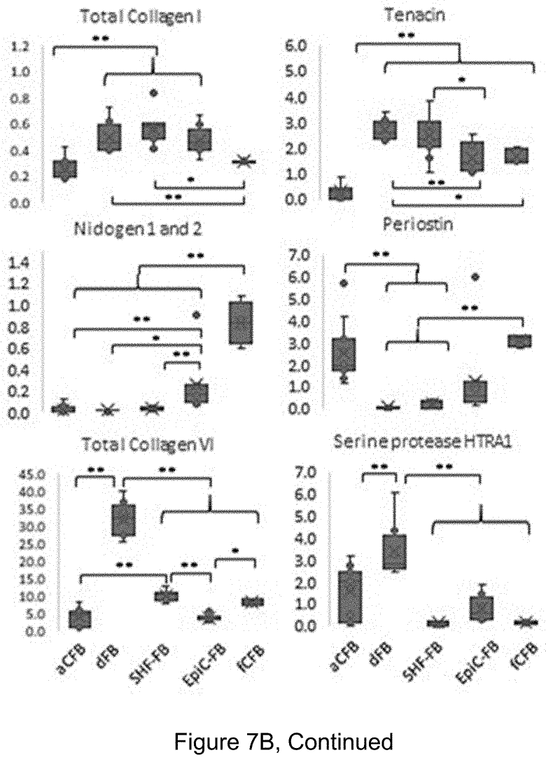

hPSCs were differentiated to CFBs through WT1⁺ epicardial cell progenitors treated with FGF2 (EpiC-FB) or through TBX1⁺HAND2⁺ second heart field progenitors (SHF-FBs) via the GiFGF protocol, as shown in FIG. 1A. Resulting CFBs from both protocols were maintained in FibroGRO media (containing 2% FBS) and passaged at ˜80% confluency. EpiC-CFBs have a similar morphology, growth rate, and time to senescence compared to SHF-FBs (FIG. 7).

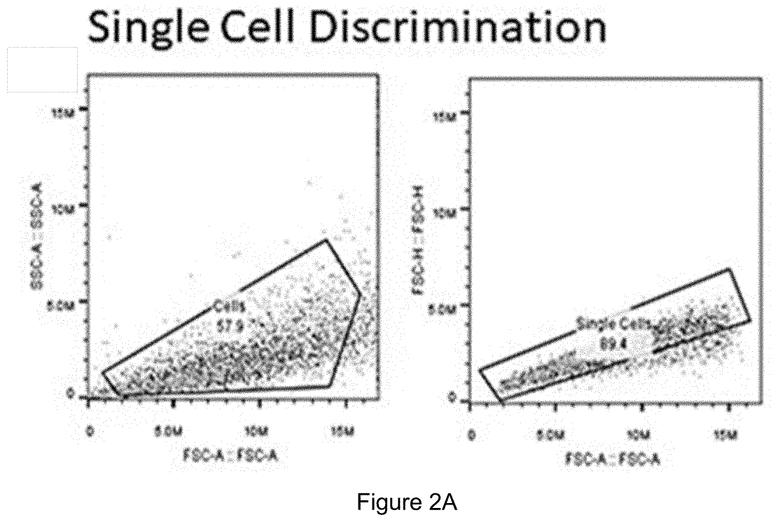

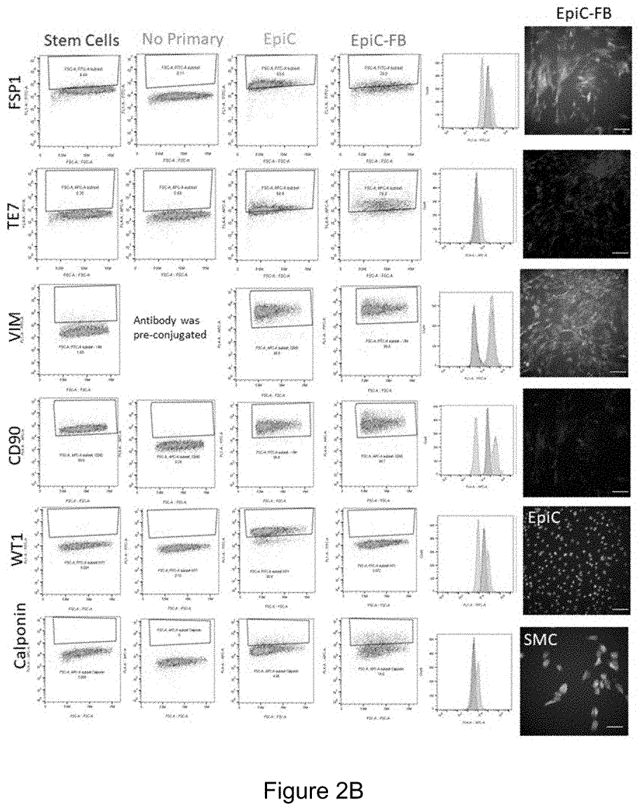

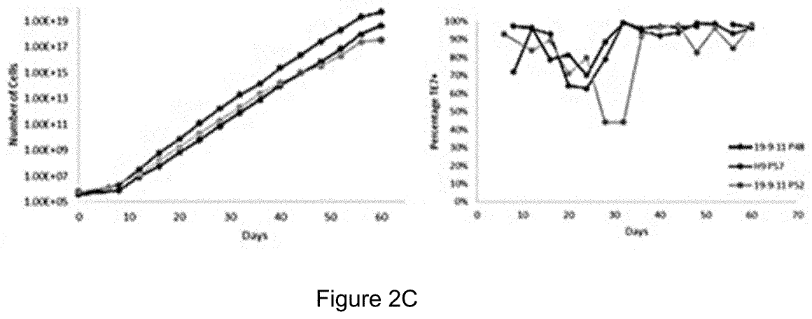

First, expression of fibroblast markers were compared in EpiC-FBs and SHF-FBs immediately after differentiation. It was observed by flow cytometry that the majority of EpiC-FBs and SHF-FBs expressed TE7, CD90, and VIM, and 20-40% of EpiC-FBs and SHF-FBs expressed FSP1, similar to primary adult dermal fibroblasts (dFBs) (FIG. 2). Surprisingly, WT1⁺ EpiCs also expressed FSP1, TE7, CD90, and VIM at similar levels to EpiC-FBs and SHF-FBs (N.S., p>0.05) (FIG. 2). The hPSC-derived CFBs did not express the epicardial marker WT1 (p<0.01 in comparison to EpiCs) or high levels of the smooth muscle cell marker calponin, consistent with a fibroblast molecular signature. Immunocytochemistry for these markers demonstrated expected nuclear localization WT1 in EpiCs, striated patterns of calponin in EpiC-SMCs, localization of VIM to the filaments in hPSC-CFBs, cell-surface localized expression of CD90 in hPSC-CFBs, and cytoplasmic localization of TE7 and FSP1 in hPSC-CFBs (FIG. 2).

The expression of a panel of cardiac transcription factors in differentiated SHF-FB and EpiC-FB were compared (FIG. 1B). Pan cardiac heart field markers ISL1 and GATA4 were expressed in EpiC-FB and SHF-FB, which aligns with previous data demonstrating protein level expression of these key cardiac markers in the SHF-FB. Expression of cardiac transcription factors and fibroblast markers in single cell sequencing data from fetal human hearts which also contained spatial data were analyzed (Asp et al., 2019, Cell. 179, 1647-1660.e19). Asp and others identified five fibroblast clusters based upon their high expression of ECM proteins and lack of sarcomeric gene expression. Here, it was verified that these fibroblast clusters contained cells which expressed high levels of the cardiac transcription factor GATA4 and fibroblast marker THY1 and VIM and then proceeded using these clusters for the analysis set forth herein. TBX2 was expressed in cells identified as related to larger vessel development by gene ontology analysis (Cluster 8), TBX3 was expressed in cells within the outflow tract (Cluster 5) but not those associated with the base of the outflow tract (Cluster 2), and TBX18 was expressed in the atrioventricular sub-epicardial mesenchyme (Cluster 3).

qPCR analysis showed that EpiC-FBs expressed significantly higher levels of HAND2 (p<0.01), TBX18 (p<0.01), and TBX20 (p<0.01), which are expressed in the epicardium and epicardial-derived cells, compared to the SHF-FBs (FIG. 1B). EpiC-FBs also expressed higher levels of TBX3 (p<0.01), which is important in conduction system development and TBX2 (p<0.01), a marker associated with the outflow tract and atrioventricular canal development. TBX1 is a transient transcription factor expressed during second heart field development, and has previously been shown to be upregulated in SHF progenitors during differentiation to SHF-FB. Compared to EpiC-FBs, differentiated SHF-FB also expressed higher levels TBX1 (p<0.05). This suggests the hPSC differentiation protocols can generate fibroblast populations expressing distinct sets of markers which are representative of in vivo cardiac fibroblast populations.

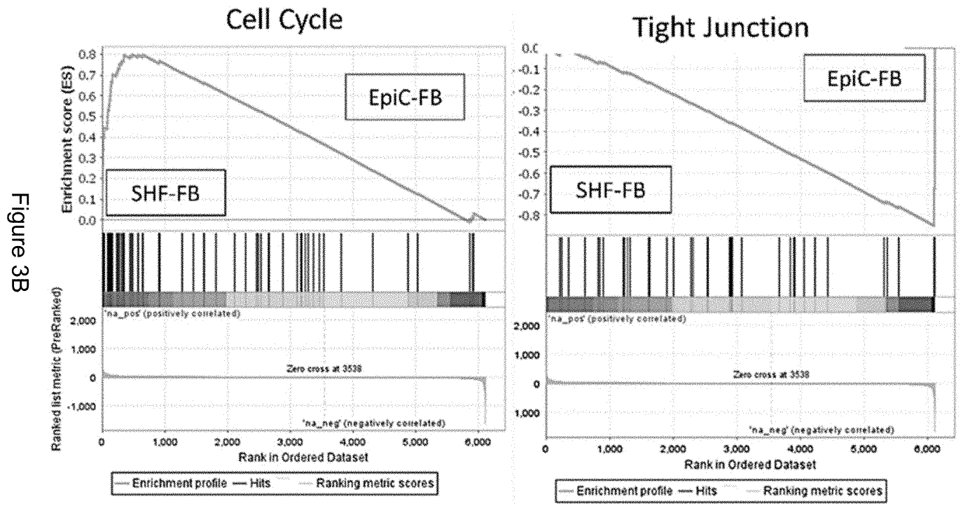

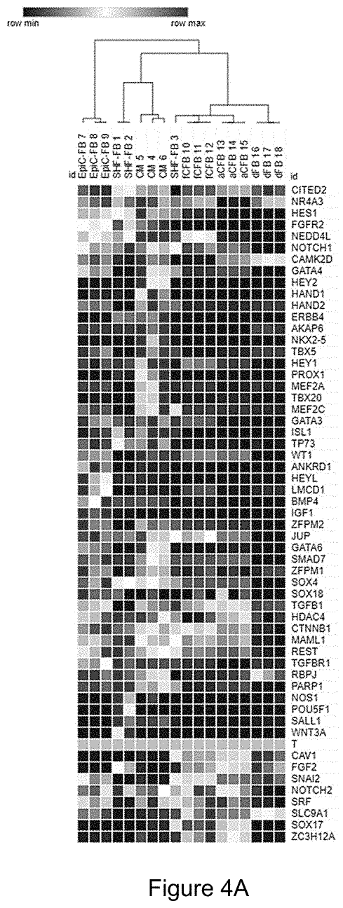

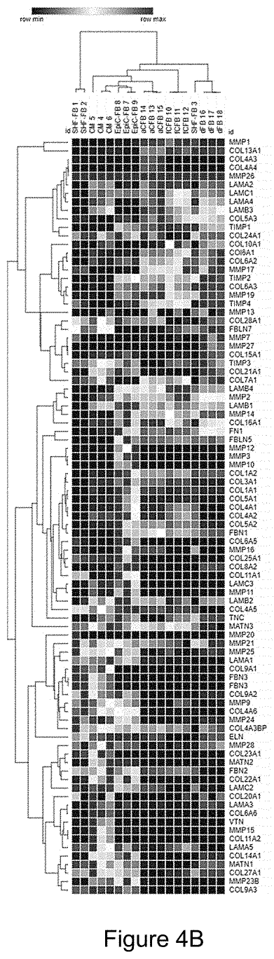

To further probe molecular similarities and differences between CFB populations, bulk RNA sequencing was performed on SHF-FBs, EpiC-FBs, primary human fCFBs, primary human aCFBs, and primary adult dFBs. Three independent differentiation replicates were used in H9 hESCs for the hPSC-derived cell types and three technical replicates of the primary cell types. Although significant batch-to-batch variation was observed between the SHF-FB differentiations, all samples expressed fibroblast markers VIM, THY1, and DDR2, and the cardiac transcription factor GATA4 to similar levels. Between the different fibroblast populations, differential expression of cardiac transcription factors was observed and ECM related proteins (FIG. 4), which emphasizes fibroblast transcriptomic heterogeneity amongst and within tissues. The data suggested that fCFB and aCFB samples were primarily from the epicardial lineage based on high expression of TBX3 and TBX18, and therefore, these samples likely predominantly represent one CFB subtype. However, both SHF-FBs and EpiC-FBs expressed pan-cardiac markers ISL1 and GATA4 at a similar level to primary CFBs, suggesting that the hPSC-CFB populations recapitulated some degree of the primary CFB molecular signature.

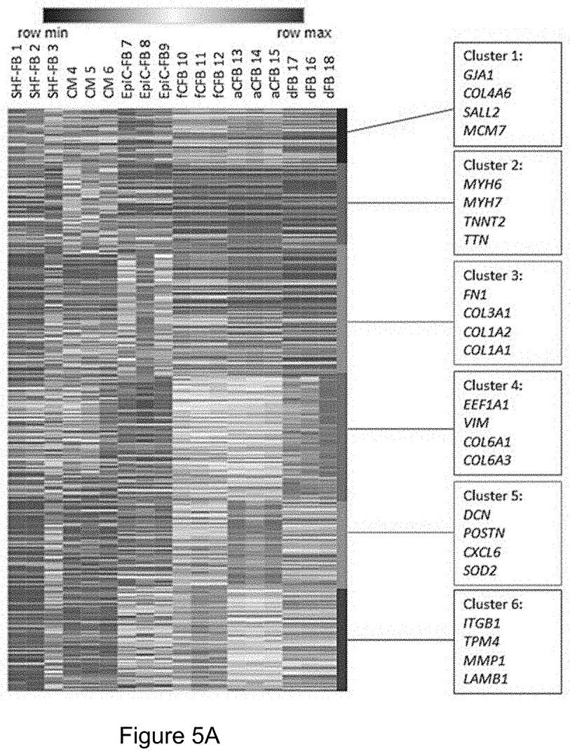

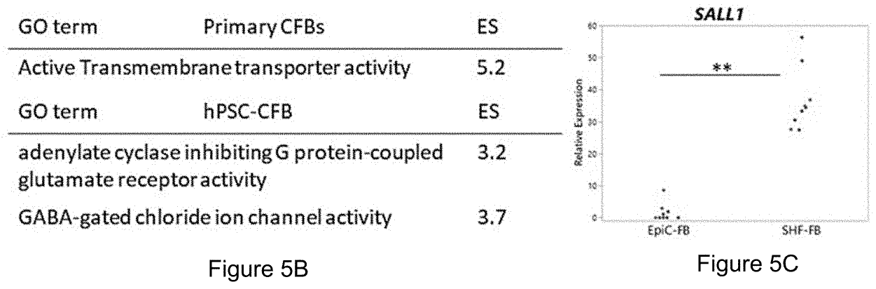

k-means clustering was performed on the top 1,000 differentially expressed genes and identified clusters of genes enriched in each cell type (FIG. 5). Cluster 2 (CM-specific) included sarcomeric genes such as MYH6, MYH7, TNNT2, and TTN which demonstrate that CMs are distinct from the fibroblast samples as expected. Additionally, COL6A1 and COL6A3 were enriched in the dFBs (Cluster 4) which suggested a different composition of ECM compared to CFB. Cluster 1 (genes enriched in SHF-FB) included genes such as GJA1, known to be important in fibroblast-CM interconnectivity, and MCM7, important in cell cycle regulation. Cluster 3 (genes enriched in EpiC-FB) included ECM proteins FN1, COL3A1, COL1A1, and COL1A2 suggesting a distinct composition of ECM between the EpiC-FB and SHF-FB. Clusters 4, 5, and 6 were genes enriched in primary CFB and dFB and included FB markers such as VIM, DCN, and POSTN. Gene ontology analysis of differentially expressed genes yielded active transmembrane transporter activity in primary CFBs and adenylate cyclase inhibiting G protein-coupled glutamate receptor activity and GABA-gated chloride ion channel activity in hPSC- CFBs which has previously been identified as present in the heart. This suggested that hPSC-CFBs might be representative of an earlier stage in development than primary CFBs samples.

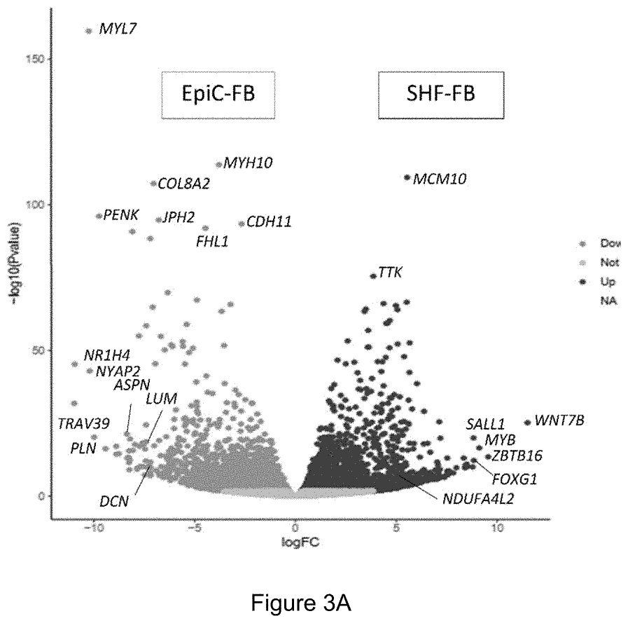

A direct comparison of SHF-FB and EpiC-FB transcriptomes, shown in FIG. 3A, highlighted key molecular differences which have been linked to specific developmental lineages. For example, MYH10 was more highly enriched in the EpiC-FBs than in SHF-FBs, and has been shown to inhibit epithelial-to-mesenchymal transition of epicardial cells during mammalian development. Additionally, Sall1 is expressed in precardiac mesoderm and expression is maintained in the murine second heart field, and Wnt7b is expressed in the remodeling atrioventricular and mitral valve at E16.5 (Morita, et al. J. Mol. Cell. Cardiol. 92, 158-162 (2016); Alfieri, et al. Dev. Biol. 338, 127 (2010). Differential expression of SALL1 was validated in three independent differentiations and demonstrated that indeed this is a marker of the SHF-FB (FIG. 5C). Additionally, comparing key markers of CFB subtypes from a human spatiotranscriptomic dataset, DCN, LUM, PENK, and ASPN were enriched in the EpiC-FBs and NDUFA4L2 was enriched in the SHF-FBs (Asp, et al., Cell. 179, 1647-1660.e19 (2019)). GSEA analysis of differentially expressed genes using KEGG pathways identified terms such as cell cycle upregulated in SHF-FBs and gap junctions in EpiC-FBs (FIG. 3C). Taken together, these data suggested that hPSC-derived SHF-FBs and EpiC-FBs are transcriptionally similar to primary CFBs and expressed distinct molecular signatures corresponding with their developmental lineages.

### Example 2

### Mass Spectrometry of Decellularized Matrix reveals hPSC-CFB Lineage Leads to a Distinct Matrix Composition

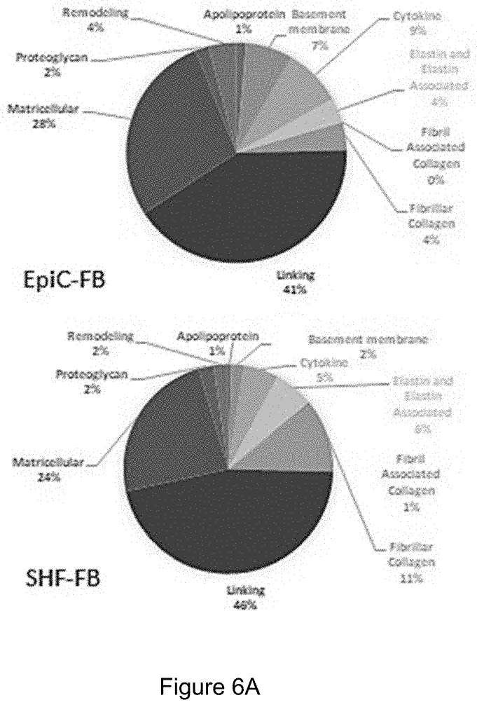

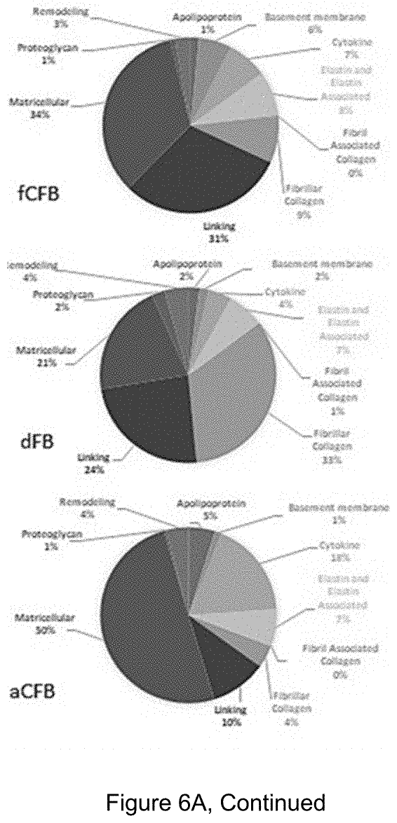

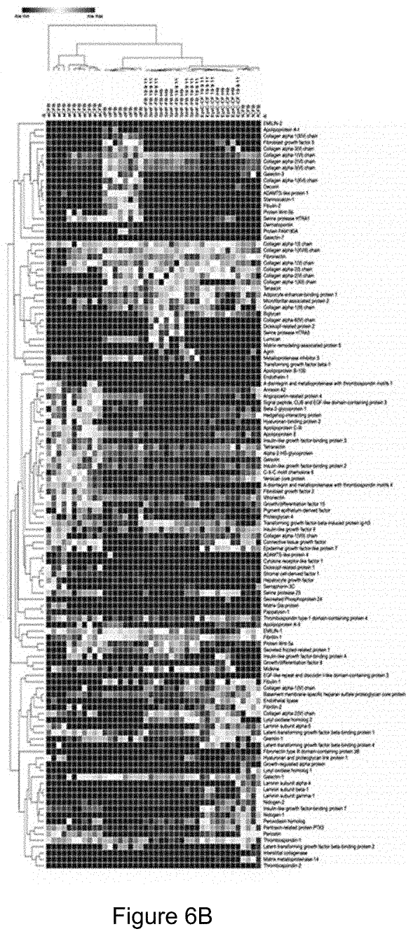

Transcriptomic analysis identified differentially expressed ECM and ECM-related genes, however transcription is upstream of matrix secretion and accumulation. Therefore, to compare ECM composition secreted by EpiC-FBs and SHF-FBs on a protein level, mass spectrometry was performed on decellularized matrix after ten days of high-density culture. Validation that the matrix was decellularized was done by Hoechst staining (FIG. 7A). To compare the overall composition of deposited ECM, proteins were divided into ten distinct categories (FIG. 6A). Notably, fCFBs and the hPSC-CFBs showed similar relative percentages of all ten categories, suggesting that the hPSC-CFBs have a more fetal-like phenotype. The most prevalent ECM category for fCFBs and hPSC-CFBs was linking ECM, composed primarily of fibronectin. By contrast, the most prevalent ECM category in aCFBs was matricellular proteins, ECM components not typically involved in structural support but that instead interact directly with bioeffector molecules. dFB matrices were most distinct from CFB due to higher accumulation of fibrillar proteins, including collagen III and VI, consistent with transcriptomic analysis (FIG. 7B). Collagen VI is abundant in the native dFB matrix and is important in dFB matrix assembly and regulating cell motility, whereas in the heart, collagen VI is only present under high stress conditions. dFB matrices also exhibited differences in remodeling proteins including high proportions of serine protease HTRA1.

Hierarchical clustering of all 118 proteins detected showed low sample technical and biological variation and no dependence on hPSC line used to generate the CFBs (H9 hESC or 19-9-11 hiPSC). hPSC-CFB matrices clustered with fCFB matrices (FIG. 6B) consistent with the ECM category analysis of FIG. 6A. fCFBs and hPSC-CFB matrices clustered separately from both aCFB and dFB matrices largely based upon deposition of greater amounts of fibronectin (FIG. 7B). In the developing mouse heart, fibronectin has been shown to regulate cardiovascular morphogenesis through integrin signaling, and in vitro coculture of CFBs and CMs isolated from E12.5-E13.5 hearts promoted CM proliferation through fibronectin synthesis. Surprisingly, the dFB matrix clustered more closely to fCFB and hPSC-CFB matrices than the aCFB matrix. Among various matricellular proteins that were detected at high levels in the aCFB matrix were insulin-like growth factor binding proteins and angiopoietin-related protein 4, suggesting these proteins could play a role in homeostasis of adult heart (FIG. 7B). Notably, insulin-like growth factor binding proteins have been studied as biomarkers for cardiovascular disease risk in adults.

Comparing the compositions of matrices deposited by the fCFBs and hPSC-CFBs with aCFBs from another dataset, higher levels of fibronectin (p<0.01), collagen I (p<0.01 comparing hPSC-CFB to aCFB) and tenascin (p<0.01) were observed in the hPSC-CFBs (FIG. 7B). Additionally, decreased lysyl oxidase was observed, a key remodeling protein that is integral for elastin and collagen crosslinking and fibrillogenesis, in aCFB matrices relative to fCFB (p<0.01), SHF-FB (p<0.05) and EpiC-FB (p<0.01) matrices. This was consistent with the substantial amount of collagen and elastin synthesis and remodeling that occur early in development and in the first few weeks postnatally. Higher levels of Collagen I in the hPSC-CFB matrices were observed compared to aCFB matrices (p<0.01) while overall low levels of collagen III were detected across all CFB matrices. Taken together, these results demonstrated that CFB matrix composition changes as the heart matures. hPSC-CFB and fCFB matrices contain higher levels of fibronectin (p<0.01), lysyl oxidase (p<0.01), and higher levels of collagen I (p<0.01) compared to the aCFB matrices, which suggested they are more fetal than adult-like.

Additionally, there were a few significant differences between the EpiC-FB and fCFB matrices relative to the SHF-FB matrices indicating a lineage-dependent matrix composition for EpiC-FB and SHF-FB. One major difference, as seen in FIG. 6A, is the large amount of basement membrane proteins present in EpiC-FB and fCFB matrices. This difference can be partially attributed to a slightly larger abundance of laminins and collagen IV in EpiC-FB and dFB matrices but is mainly due to large percentage of basement membrane-specific heparan sulfate proteoglycan core protein, also known as perlecan, present in EpiC-FB and fCFB matrices (p<0.01 in comparison to all other matrices). Perlecan has been shown to play an integral role in development of the coronary vasculature, heart stability as well as cardiomyocyte organization and sarcomere structure. These data suggested that EpiC-FB can play a role in perlecan synthesis during development. Other matrix components detected in larger proportions in EpiC-FB and fCFB matrices include pentraxin related protein PTX3 (N.S. EpiC-FB matrix compared to aCFB matrix, all other comparisons p<0.01) and Nidogen 1 and 2 (p<0.05) (FIG. 7B). Interestingly, periostin was present in larger proportions in aCFB matrices (p<0.01) as well as fCFB (p<0.01) matrices in comparison to SHF-FB matrices suggesting a lineage-specific role of periostin in development and adult cardiac matrix homeostasis.

### Example 3

### hPSC-CFBs Secrete Lineage Specific Factors

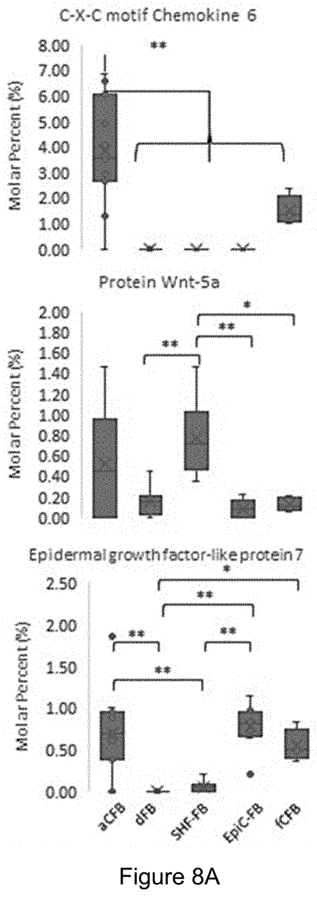

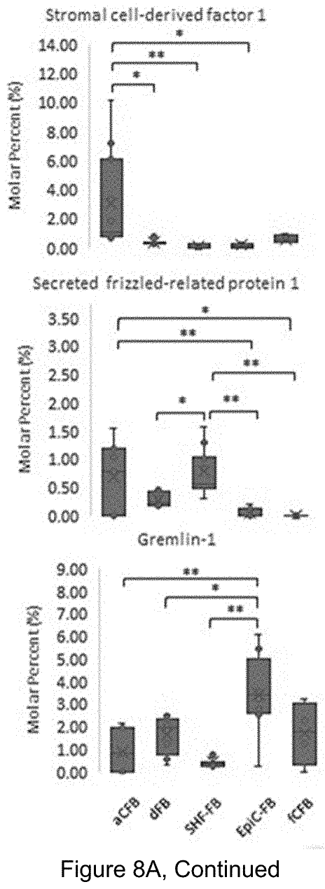

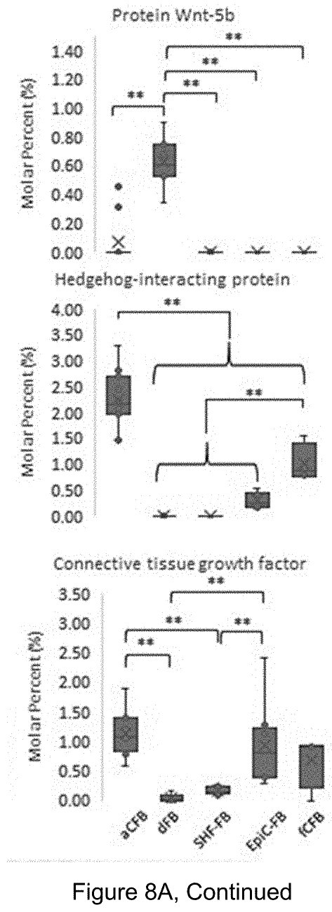

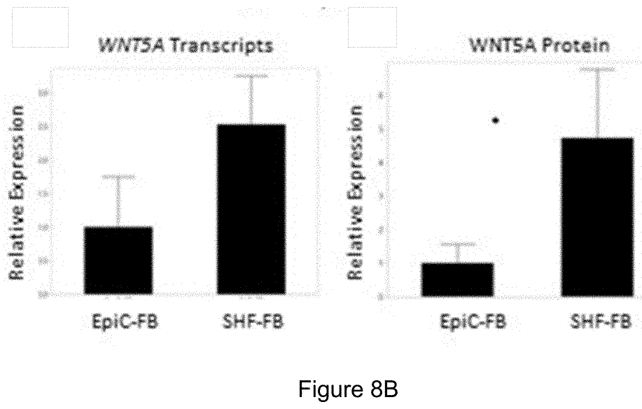

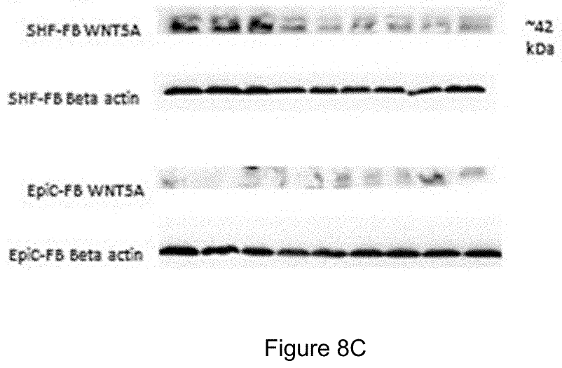

CFBs secrete signaling factors that have been shown to alter CM contraction through ion channel remodeling and cardiac hypertrophy in vitro. Several differentially upregulated secreted factors were identified in the aCFB matrices compared to the fCFB matrices, including C-X-C motif Chemokine 6 (p<0.01) and Growth/differentiation factor 15 (p<0.01) (FIG. 8B). These factors were higher in the aCFB matrices compared to hPSC-CFB matrices (C-X-C motif Chemokine 6 p<0.01, stromal cell-derived factor 1 p<0.05, Dickkopf-related protein 1 p<0.01, and Growth/differentiation factor 15 p<0.01) and contributed to distinct clustering of aCFB matrices relative to other CFB matrices, again suggesting that hPSC-CFBs were more fetal-like than adult-like. There were high proportions of secreted hedgehog-interacting protein, a known inhibitor of the hedgehog signaling pathway, associated with aCFB and fCFB matrices (p<0.01 in comparison to all other matrices). SHF-FB matrices contained low levels of Gremlin-1 (p<0.01 in comparison to EpiC-FB matrices), epidermal growth factor-like protein 7 (p<0.01 in comparison to EpiC-FB and aCFB matrices), and connective tissue growth factor (p<0.01 in comparison to EpiC-FB and aCFB matrices) which have been shown to be upregulated during cardiac fibrosis, compared to EpiC-FB and aCFB matrices.

### Example 4

### Fibroblast Activation Revealed Greater Activation Potential in EpiC-FBs than SHF-FBs

Extracellular matrix production is a key function of CFBs, however they also play key roles in tissue development, maintenance, and repair. Two functional assays were performed to ascertain the ability of the hPSC-CFBs to become activated under stress.

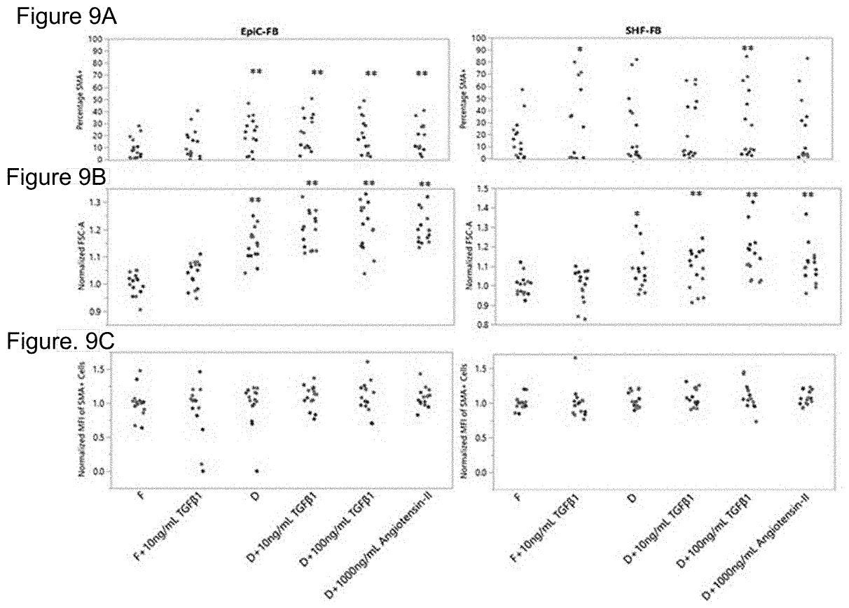

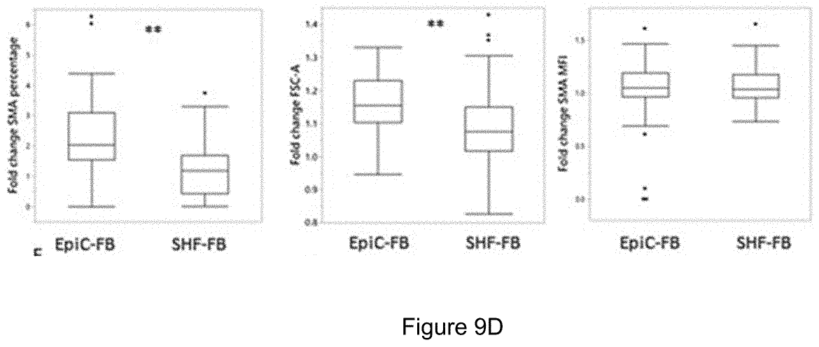

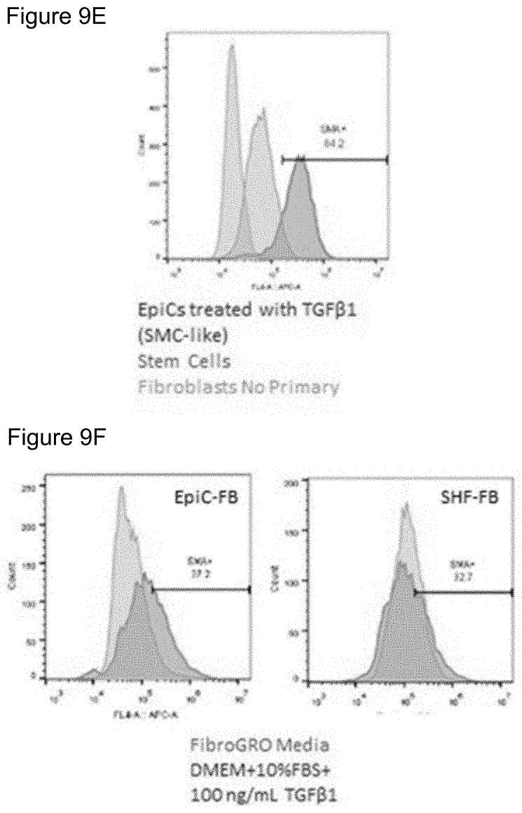

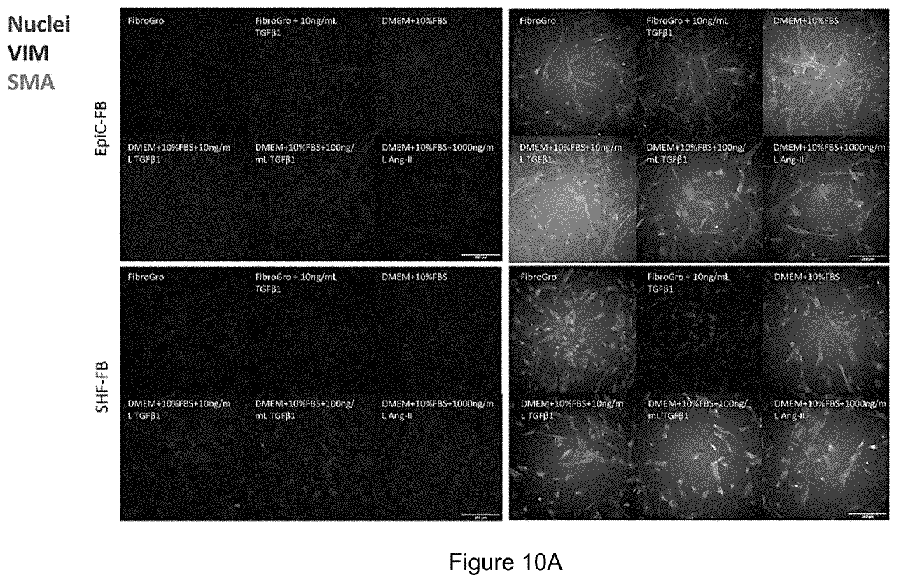

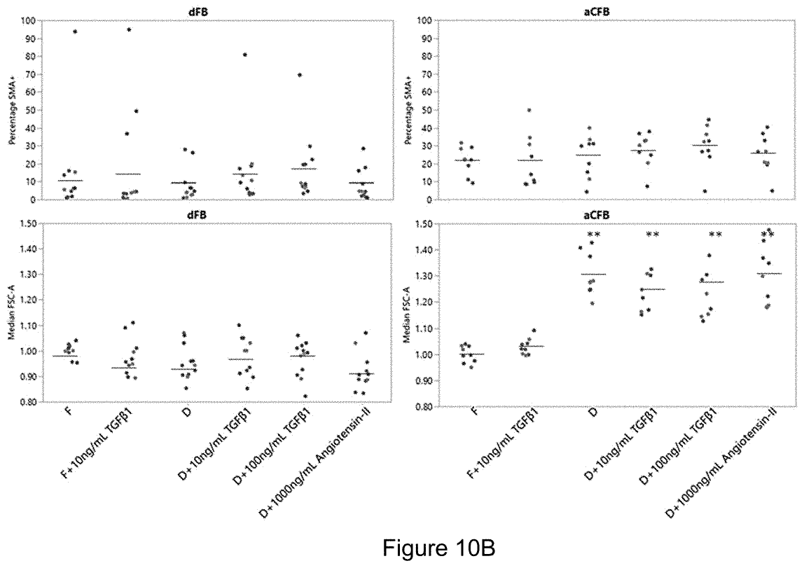

Fibroblasts when stressed in vivo transition to a myofibroblast state characterized by an increased cell size and increased expression of smooth muscle actin (SMA). To test if hPSC-derived SHF-FBs and EpiC-FBs exhibited differential activation in vitro, these cells were treated with TGFβ1-, Angiotensin-II-, and FBS-supplemented media for 2 days and then compared by flow cytometry wherein theisrelative cell sizes were assessed by forward scatter area (FSC-A) and SMA expression. Undifferentiated hPSCs were used and no primary antibody conditions as negative flow gating controls and EpiC-SMCs as a positive control. FibroGRO basal media (F) was used as the control since it is a commonly used maintenance media with minimal activation and individually tested the effects of TGFβ1, Angiotensin-II, and serum. Across multiple differentiations, fibroblast activation was observed as demonstrated by SMA induction and increased FSC-A in EpiC-FBs (F+10 ng/mL TGFβ1 N.S, all others p<0.01 for change in FSC-A and SMA expression), SHF-FBs (F+10 ng/mL TGFβ1 N.S change in FSC-A and p<0.05 change in SMA expression, D p<0.05 change in FSC-A and N.S. change in SMA expression, D+10 ng/mL TGFβ1 and D+1000 ng/mL Angiotensin-II p<0.01 change in FSC-A and N.S. change in SMA expression, D+100 ng/mL TGFβ1 p<0.01 change in FSC-A and p<0.01 change in SMA expression), and aCFBs (F+10 ng/mL TGFβ1 N.S. change in FSC-A, all other conditions p<0.01 change in FSC-A) and not dFBs (N.S. change in FSC-A or SMA expression) under serum or serum with the addition of TGFβ1 and Angiotensin-Il (FIG. 4, FIG. 10). A high variability in basal SMA activation levels was observed between hPSC-CFB differentiated from different stem cell lines, which could result from genetic variability as previously demonstrated, however differentiation-to-differentiation variability was observed. Regardless, higher activation levels in EpiC-FBs were observed compared to SHF-FBs (fold change SMA percentage p<0.01, fold change FSC-A p<0.01) (FIG. 9D).

### Example 5

### Fibroblast Mineralization Revealed Greater Potential in SHF-FB than EpiC-FB

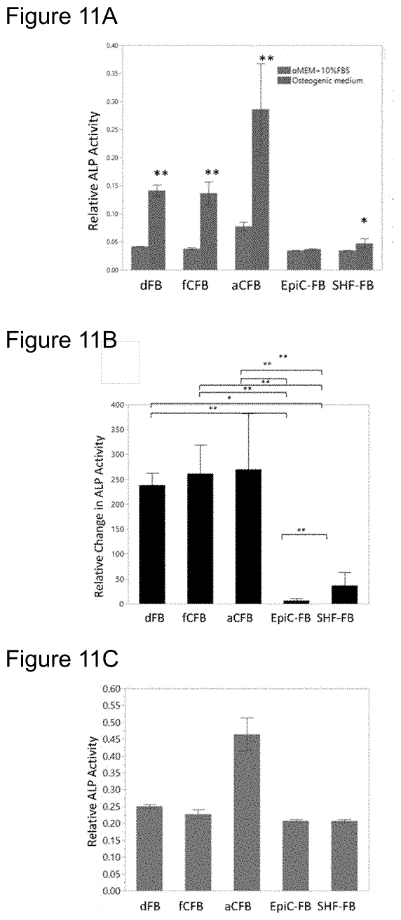

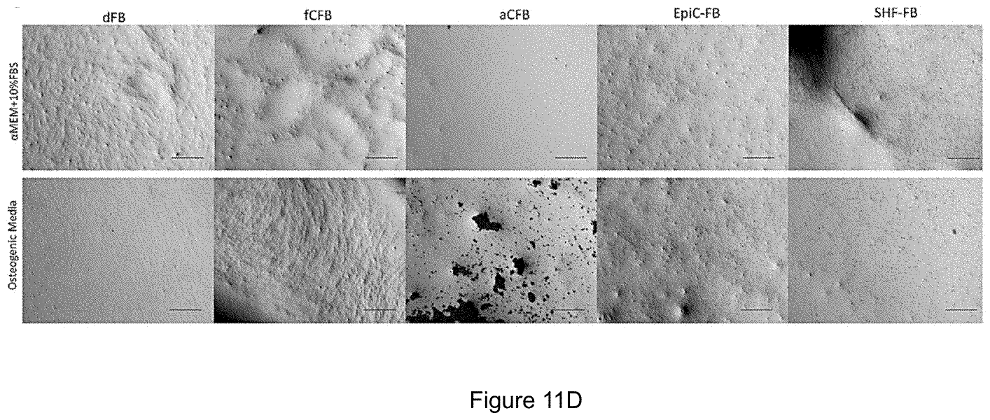

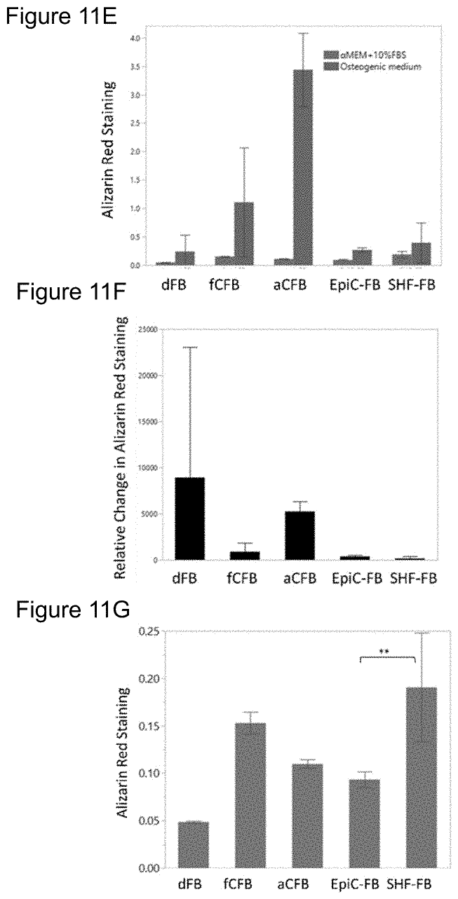

To test whether EpiC-FBs and SHF-FBs have different mineralization potentials, primary dFBs, fCFBs, aCFBs, EpiC-FBs, and SHF-FBs were cultured in osteogenic medium containing L-ascorbic acid, β-glycerophosphate, and dexamethasone for four weeks and changes in ALP activity and Alizarin red staining compared. Increased ALP activity was observed upon addition of osteogenic factors in all cell types except EpiC-FBs (FIG. 11A). Additionally, hPSC-CFBs had lower mineralization potential, as measured by percentage change in ALP activity with addition of the osteogenic factors, compared to primary fibroblasts (p<0.05), and across four differentiations, EpiC-FBs had lower osteogenic potential than SHF-FBs (p<0.01) (Figure5B). To assay mineralization, Alizarin red was used to stain and quantified fluorescence. Staining was highest in the aCFBs when exposed to osteogenic factors, and no difference was observed between SHF-FBs and EpiC-FBs (FIGS. 5D-5F). Overall, this suggests that SHF-FBs may have a higher calcification potential compared to EpiC-FBs.

Calcification, mineralization, and nodule formation have been used to analyze valve interstitial cell activation level. To compare if hPSC-CFBs have differential response to activation, the cells were treated in serum-supplemented media, which caused fibroblast activation, for four weeks. Over multiple differentiations, higher ALP activity (p<0.05) was observed and Alizarin red staining (p<0.01) in the SHF-FBs compared to EpiC-FBs (FIGS. 5C and 5G). Taken together with the previous results, this suggested that EpiC-FBs-FBs had lower mineralization and calcification potential in response to serum with or without additional osteogenic factors

### Example 6

### Cardiac Fibroblasts Promoted Robust Microtissue Formation and Enhance Calcium Cycling

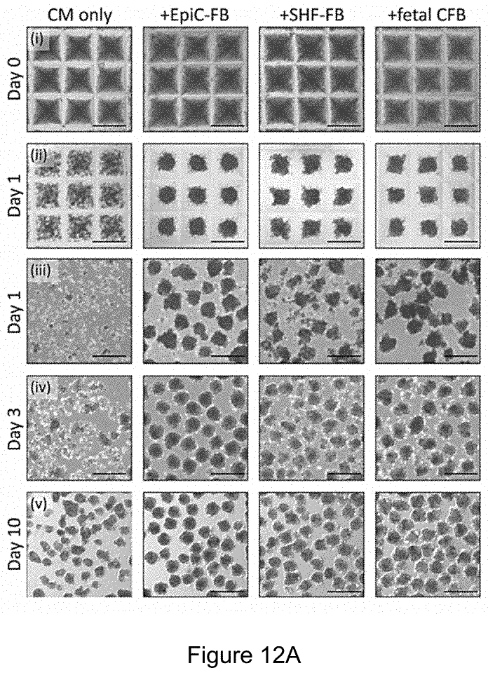

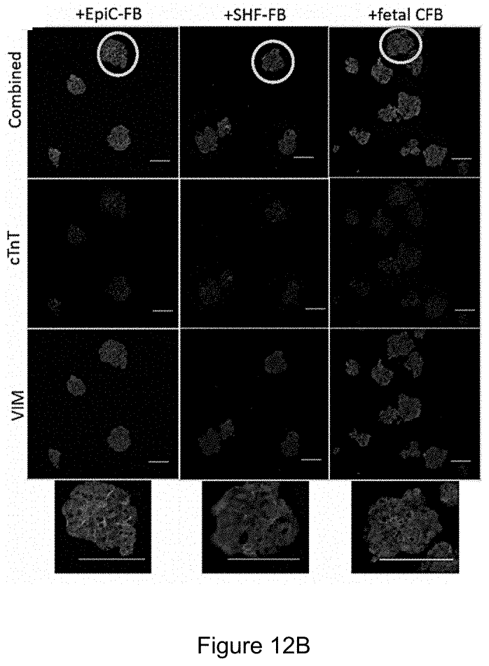

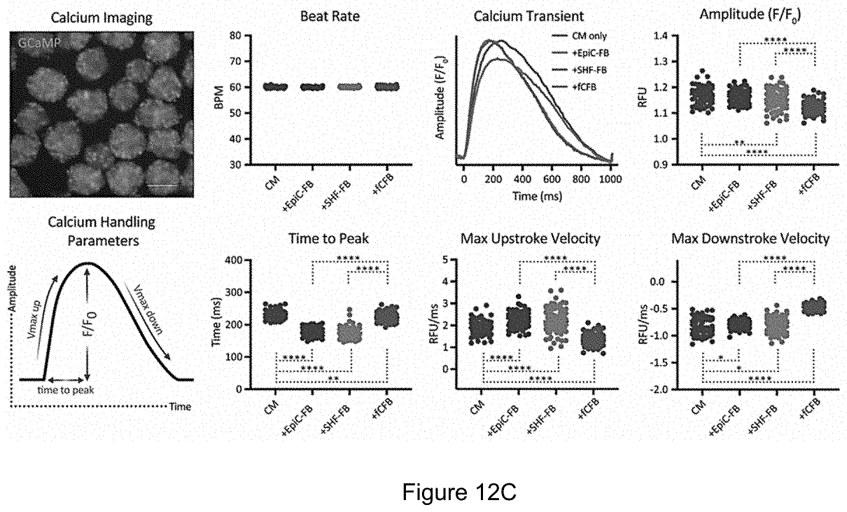

Although cell level analysis provided insight into fibroblast function, it did not address how CFBs behave in cardiac tissues. Therefore, engineered cardiac microtissues were formed from a heterotypic mixture of hPSC-CMs and CFBs in order to assess how EpiC-FBs and SHF-FBs supported CM function compared to primary human fCFBs. The same pool of lactate-enriched CMs was used for all microtissue groups, suggesting that any differences in tissue formation and function were due to the different sources of fibroblasts and their ability to interact with CMs. The population of highly-enriched CMs did not form into uniform microtissues when aggregated alone (without CFBs), but instead clumped into small clusters (FIG. 12A). In contrast, all microtissues that contained CFBs self-assembled into 3D spheroids within 24 hours of seeding into microwell molds (FIG. 12A), indicating that a stromal component is necessary to facilitate robust microtissue assembly. These heterotypic cardiac microtissues compacted after removal from the microwell molds, as demonstrated by more distinct tissue boundaries and increasingly spheroidal shape 3 days after tissue formation compared to 1 day after, and stably persisted throughout the 10 days of microtissue culture. The “CM only” control microtissues appeared more stable with increased culture duration, likely because those that persisted throughout culture contained the small fraction of non-myocyte cells that carried over from the CM differentiation and enrichment. Sectioning of cardiac microtissues showed CFBs dispersed amongst CMs by staining for VIM and cTnT, respectively (FIG. 12B).

Calcium handling properties of engineered cardiac microtissues containing SHF-FBs or EpiC-FBs were assessed in order to determine whether different CFB subtypes altered CM function. Day 10 microtissues were subjected to 1 Hz electrical field stimulation in order to eliminate intrinsic differences in beat rate between the individual tissues (FIG. 12C). Video-based imaging of the fluorescence intensity of the genetically-encoded calcium sensor GCaMP6f in the CMs enabled quantification of the kinetic parameters of the microtissue calcium transients. There was no statistical significance between any of the calcium handling properties from microtissues made with EpiC-FBs compared to those made with SHF-FBs, indicating that the hPSC-CFB subtypes similarly supported hPSC-CM function. The calcium transient amplitudes of the +EpiC-FB and +SHF-FB microtissues were similar to those of the hPSC-CM only (N.S. +EpiC-FB to CM, p<0.01 +SHF-FB to CM) microtissues and slightly higher than the values of the microtissues containing primary fCFs (p<0.0001) (FIG. 12C). The microtissues comprised of the hPSC-CFBs displayed the fastest upstroke kinetic properties, taking the shortest time to reach the peak of the calcium transient (p<0.001 in comparison to CM or +fCFB) and exhibiting the fastest maximum upstroke velocities (p<0.001 in comparison to CM or +fCFB). The maximum downstroke velocities followed the same trend as the amplitude values, where the CM only, CM+EpiC-FB, and CM+SHF-FB microtissues exhibited similar velocity values and were all faster than the CM+fCFB microtissues (p<0.0001).

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description can be made without departing from the spirit or scope of the present invention, as defined in the following claims.

1. : A method for generating a population of cardiac fibroblast cells, the method comprising:
culturing epicardial progenitor cells in a culture medium comprising a fibroblast growth factor, whereby a cell population comprising cardiac fibroblast cells is obtained.

2. The method of claim 1, wherein the epicardial progenitor cell is human.

3. The method of claim 1, wherein the culture medium is serum-free.

4. The method of claim 1, wherein the cell population comprising cardiac fibroblast cells is obtained after about 10 days in culture.

5. The method of claim 1, wherein the fibroblast growth factor is bFGF.

6. The method of claim 5, wherein bFGF is present at a concentration of about 1 ng/mL to about 1000 ng/mL.

7. The method of claim 1, wherein at least 90% of cells in the cell population are cardiac fibroblast cells positive for expression of one or more markers selected from TBX2, TBX18 and TBX20.

8. The method of claim 1, wherein the cardiac fibroblast cells obtained are capable of undergoing expansion for about 60 days, wherein the cardiac fibroblast cells maintain expression of TE-7, vimentin, and/or for GATA4 for the about 60 days.

9. The method of claim 1, wherein the cardiac fibroblast cells are capable of undergoing at least 15 cell passages.

10. A population of cardiac fibroblast cells produced by the method of claim 1.

11. The population of cells of claim 10, wherein at least 90% of cells in the cell population are cardiac fibroblast cells positive for expression of one or more markers selected from TBX2, TBX18 and TBX20.

12. A method of screening a test agent, the method comprising:
(a) co-culturing the population of cardiac fibroblast cells prepared according to the method of any one of claims 1-9 and the test agent;
(b) measuring a functional parameter of the contacted co-culture; and
(c) comparing the functional parameter to that parameter measured in a co-culture which has not been contacted with the test agent, wherein modulation of the functional parameter after contact with the test agent indicates the test agent is a candidate therapeutic agent.

13. The method of claim 12, wherein the test agent is (i) an organic compound; (ii) a nucleic acid; (iii) a peptide; (iii) a polypeptide; or (iv) an antibody.

14. The method of claim 12, wherein the test agent is an antifibrotic therapeutic agent.

15. The method of claim 12, wherein the functional parameter is electrical impulse propagation pattern, conduction velocity, or action potential duration.

16. A kit for differentiating epicardial progenitor cells into cardiac fibroblasts, the kit comprising: (i) a culture medium suitable for differentiating epicardial progenitor cells into cardiac fibroblasts; (ii) a fibroblast growth factor; and (iii) instructions describing a method for generating cardiac fibroblasts, the method employing the culture medium and the fibroblast growth factor.
